(19) United States
(12) Patent Application Publication
(10) Pub. No.: US 2009/0221651 A1
(43) Pub. Date: Sep. 3, 2009

(54) CHEMICAL PRECONDITIONING AS A PREVENTATIVE OR TREATMENT FOR EXCITOTOXIC SYNAPTIC DAMAGE

(76) Inventors: Matthew J. Bellizzi, Rochester, NY (US); Shao-Ming Lu, Setauket, NY (US); Harris A. Gelbard, Pittsford, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/092,110

(22) PCT Filed: Oct. 30, 2006
(86) PCT No.: PCT/US06/42213

§ 371 Date: Jan. 8, 2009

Related U.S. Application Data
(60) Provisional application No. 60/731,742, filed on Oct. 31, 2005.

(51) Int. Cl. A61K 31/4406; C12Q 1/02; A61P 25/00

(52) U.S. Cl. 514/355; 435/29

(57) ABSTRACT

A method of preventing HIV-1 associated dendritic pathology in a brain cell, comprising contacting the cell with a therapeutically effective dose of a mitochondrial ATP-sensitive potassium channel agonist. A method of preventing HIV-1 associated dendritic pathology in a brain cell, comprising contacting the cell with a therapeutically effective dose of an inhibitor of succinate dehydrogenase. A method of preventing HIV-1 associated dendritic pathology in a brain cell, comprising contacting the cell with a therapeutically effective dose of a stimulator of production of reactive oxygen species. A model for the study of HIV-1 associated dendritic pathology, comprising a) contacting a hippocampal slice with platelet-activating factor; and b) stimulating the hippocampal slice of a) with high frequency stimulation.

### CROSS-REFERENCE TO RELATED PATENT APPLICATION

This application claims the benefit, pursuant to 35 U.S.C. §119(e), of the provisional U.S. patent application Ser. No. 60/731,742 filed Oct. 31, 2005, entitled “Chemical Preconditioning as a Preventative or Treatment for Excitotoxic Synaptic Damage,” which application is hereby incorporated by reference in its entirety and made a part hereof.

This work was supported by grants from the US National Institutes of Health (MH64570, MH56838 and NS31492 to M.J.B., S.M.L. and H.A.G.; AI49815 and GM07356 M.J.B.; and MH59745, MH45294 and MH62962 to E.M.). Thus, the Government has certain rights in the invention.

### BACKGROUND OF THE INVENTION

Neurologic impairment in patients with HIV-1-associated dementia (HAD) correlates well with injury to dendrites and synapses (1) but poorly with neuronal loss (2, 3). Similar results have been found in Alzheimer disease (4, 5), and dendritic injury in both diseases is characterized by focal swelling or beading, loss of spines, and reductions in overall dendritic and synaptic areas (6-9). Consequently, synaptic protection represents an area of considerable therapeutic interest.

How HIV-1 causes dendritic injury is not well understood. HIV-1 infects neurons rarely, if at all, but predominantly infects macrophages and microglia in the brain and triggers release of inflammatory mediators including HIV-1 Tat and gp120, proinflammatory cytokines, arachidonic acid metabolites, and platelet-activating factor (PAF) (23).

PAF (1-O-alkyl-2-O-acetyl-sn-glycero-3-phosphocholine) is a phospholipid inflammatory mediator that plays both physiologic and pathologic roles in the brain. Produced by neurons in response to NMDA receptor activation (26), PAF increases glutamate release from presynaptic terminals (27) and can participate in long-term potentiation (LTP) of synaptic transmission (28, 29) as well as learning and memory (30, 31). PAF brain concentrations are dramatically increased in HAD (32) and other insults (33, 34) and are associated with neurotoxicity. PAF has been shown to mediate NMDA excitotoxicity (35), and high concentrations can kill neurons in an NMDA receptor-dependent manner (32, 36).

### SUMMARY OF THE INVENTION

A method of treating or preventing HIV-1 associated dendritic pathology in a brain cell is provided, comprising contacting the cell with a therapeutically effective dose of a mitochondrial ATP-sensitive potassium channel agonist (K⁺ ATP channel agonist).

Provided herein is a method of protecting a neuron from dysfunction induced by HIV-1 induced neurotoxicity comprising contacting the cell with a K⁺ ATP channel agonist.

Further provided is a method or treating or preventing HIV-1 associated dementia (HAD) in a subject in need of such treatment or prevention, comprising administering to the subject a therapeutically effective dose of a K⁺ ATP channel agonist.

A method of treating or preventing HIV-1 associated dendritic pathology in a brain cell is also provided, comprising contacting the cell with a therapeutically effective dose of an inhibitor of succinate dehydrogenase.

Further provided is a method of treating or preventing HIV-1 associated dendritic pathology in a brain cell, comprising contacting the cell with a therapeutically effective dose of a stimulator of production of reactive oxygen species.

Further provided is a method of treating or preventing HIV-1 associated dendritic pathology in a brain cell, comprising contacting the cell with a therapeutically effective dose of a composition comprising a K⁺ ATP channel agonist and a compound selected from the group consisting of a modulator of adenosine receptor signaling and a molecule that inhibits mitochondrial hyperpolarization in a neural cell.

Further provided is a method of treating or preventing HIV-1 associated dendritic pathology in a brain cell, comprising contacting the cell with a therapeutically effective dose of a composition comprising an inhibitor of succinate dehydrogenase and a compound selected from the group consisting of a modulator of adenosine receptor signaling and an inhibitor of mitochondrial hyperpolarization in a neural cell.

Further provided is a method of treating or preventing HIV-1 associated dendritic pathology in a brain cell, comprising contacting the cell with a therapeutically effective dose of a composition comprising a stimulator of production of reactive oxygen species and a compound selected from the group consisting of a modulator of adenosine receptor signaling and an inhibitor mitochondrial hyperpolarization in a neural cell.

Further provided is a method of treating or preventing HIV-1 associated dendritic pathology in a brain cell, comprising contacting the cell with a therapeutically effective dose of a composition comprising at least two compositions selected from the group consisting of an inhibitor of succinate dehydrogenase, a stimulator of production of reactive oxygen species, a modulator of adenosine receptor signaling and an inhibitor of mitochondrial hyperpolarization in a neural cell.

Provided is a model for the study of HIV-1 associated dendritic pathology, comprising a) contacting a hippocampal slice with platelet-activating factor; and

b) stimulating the hippocampal slice of a) with high frequency stimulation.

Further provided is a composition, comprising a K⁺ ATP channel agonist and a compound selected from the group consisting of a modulator of adenosine receptor signaling and a molecule that inhibits mitochondrial hyperpolarization in a neural cell.

Further provided is a composition, comprising a K⁺ ATP channel agonist and a compound selected from the group consisting of a modulator of adenosine receptor signaling, a molecule that inhibits mitochondrial hyperpolarization, and a stimulator of reactive oxygen species in a neural cell.

Further provided is a composition, comprising an inhibitor of succinate dehydrogenase and a compound selected from the group consisting of a modulator of adenosine receptor signaling and a molecule that inhibits mitochondrial hyperpolarization in a neural cell.

Further provided is a composition, comprising a stimulator of production of reactive oxygen species and a compound selected from the group consisting of a modulator of adenosine receptor signaling and a molecule that inhibits mitochondrial hyperpolarization in a neural cell.

Further provided is a composition, comprising at least two compositions selected from the group consisting of an inhibitor of succinate dehydrogenase, a stimulator of production of reactive oxygen species, a modulator of adenosine receptor signaling and an inhibitor of mitochondrial hyperpolarization in a neural cell.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DESCRIPTION OF THE FIGURES

FIG. 1 demonstrates that cPAF reproduces dendritic pathology of HAD. (a) Golgi-stained neurons in brain tissue from patients with HAD have focal swellings and fewer dendritic spines than those from HIV-1 seropositive controls without neurologic disease. (b) Dendrites in dissociated hippocampal cultures develop similar focal swellings and decreased numbers of spines after prolonged exposure to cPAF. (c) Lower-magnification images (left, middle) show dendritic beading (arrows) accompanied by sprouting of filopodia (arrowheads) with preservation of dendrite branches in cPAF-treated cultures, and minimal change in dendrite morphology in vehicle-treated cultures. Higher-power images from the same cells (right) show dendritic spine numbers maintained in a control dendrite, and loss of spines in a cPAF-treated dendrite. (d) 56% of cPAF-treated neurons developed dendritic beading, while none of the vehicle-treated cells did (n=17, P<0.05). (e) Numbers of dendritic spines decreased by 45±5% with cPAF treatment, and remained stable in control neurons (n=10, P<0.0001). Scale bars 20 μm.

FIG. 2 illustrates dendritic injury and neuronal PAF receptor expression in HAD. In cortical tissue from patients with HAD, (a) dendritic beading and spine loss in golgi-stained neurons is associated with (b) strong PAF-R immunohistochemical staining on dendrites and neuronal cell bodies identified by co-immunostaining for MAP2. HAD tissue shows fewer MAP2-positive dendritic branches compared to tissue from HIV-1 seropositive controls, while PAF-R expression on the remaining dendrites and cell bodies is increased. (c) Higher-power field shows intense PAF-R expression on beaded dendrites in HAD compared to control dendrites. Scale bars 20 μm.

FIG. 3 demonstrates that cPAF increases vulnerability to dendritic swelling following synaptic activity. (a) In control cultures, synaptic activity due to 1 s depolarizing pulses of KCl elicited no change in dendrite morphology while 5 s pulses triggered beading throughout the dendritic arbor that recovered within 10 min. (b) In cPAF-exposed cells, 1 s pulses caused rapid dendritic beading. (c) Membrane potential recordings show bursts of action potentials and stronger, more prolonged depolarization elicited by 5 s (gray) vs. 1 s (black) KCl stimulation. (d) cPAF lowered the threshold for activity-induced dendritic beading, leading to beading in 90% of neurons following 1 s KCl pulses that caused no beading in control neurons. PAF-R antagonist BN52021 blocked the increase in vulnerability. (e) Rapid recovery of dendritic beading is prevented by NPPB, an inhibitor of regulatory volume decrease in swollen neurons. *, P<0.001. Scale bars 20 μm.

FIG. 4 shows that cPAF replaces long-term potentiation with dendritic beading in hippocampal slices. (a) Dendritic beading in a cPAF-exposed CAl pyramidal neuron 45 min after high-frequency Schaffer collateral stimulation (HFS), with no disruption of dendrite or spine morphology in following HFS in vehicle-treated cells. (b) HFS elicited dendritic beading in 11 of 19 cells from cPAF-treated slices, and in 0 of 13 cells from vehicle-treated slices (*P<0.001). PAF-R antagonists BN52021 and CV-3988 reduced dendritic beading to 1 of 10 and 1 of 7 cells, respectively (**P<0.05 vs. cPAF). (c) The amplitude and duration of post-synaptic depolarization during HFS is unaffected by cPAF exposure. (d) Excitatory synaptic transmission is strongly potentiated following HFS in vehicle-treated slices (2.66±0.44-fold relative to baseline at 40 to 50 min, n=13, P<0.001). In cPAF-treated slices, cells that did not develop dendritic beading underwent a smaller but significant potentiation (1.60±0.26 relative to baseline, n=8, P<0.05) while EPSPs in cells whose dendrites did bead were not potentiated at all (0.84±0.12 relative to baseline, n=11, P<0.01 vs. vehicle and P<0.05 vs. cPAF-treated cells without dendritic beading). Representative EPSPs from vehicle- (upper right) and beaded, cPAF-treated cells (lower right) are averages of 10 consecutive traces recorded at baseline and 50 min post-HFS. Scale bars 20 μm.

FIG. 5 shows that activity-dependent dendritic beading is delayed, long-lasting and local. Dendritic beading in a cPAF-exposed hippocampal slice develops with a delay after high-frequency stimulation (HFS), and progresses throughout the recording trial. In addition, focal swellings are restricted to discrete regions along the dendrite, with no apparent disruption of dendrite and spine morphology in intervening areas. Scale bars 20 μm.

FIG. 6 demonstrates that chemical preconditioning prevents calcium- and caspase-dependent beading and restores LTP. (a) Rates of dendritic beading and (b) EPSP potentiation following high frequency Schaffer collateral stimulation in hippocampal slices exposed to cPAF. Post-synaptic calcium chelation by intracellularly-applied BAPTA eliminated dendritic beading as well as synaptic potentiation (n=6). Post-synaptic caspase-3,6,7,9,10 inhibition by intracellular Ac-DEVD-CHO (10 μM) prevented dendritic beading, but failed to restore a lasting potentiation (n=7), while nitric oxide synthase inhibitor L-NAME had no effect on rates of dendritic beading compared with cPAF alone (FIG. 4). Pretreatment with the mitochondrial K_{ATP} agonist diazoxide prevented dendritic beading and restored LTP in cPAF-exposed slices (2.10±0.27-fold potentiation at 40 to 50 min, n=7). *, P<0.01 vs. cPAF alone (FIG. 4).

FIG. 7 shows that PAF receptor immunostaining is specific in control and HAD cortical tissue. Immunohistochemical staining for PAF-R, detected by horseradish peroxidase using either Tyramide Red (Tyr Red, upper panels) or DAB (lower panels) as fluorochrome or chromagen, respectively, is increased in cortical tissue from patients with HAD compared to HIV-1 seropositive controls. The staining pattern of PAF-R is identical to that seen in sections double stained with MAP2 antibody (FIG. 2). Pre-incubation of the PAF-R antibody with its PAF-R-derived peptide antigen virtually eliminated staining in all cases, demonstrating a specific interaction between the antibody and PAF-R in these tissues. Scale bar 20 μm.

FIG. 8 demonstrates that PAF receptor antagonists do not restore LTP in cPAF-exposed slices. High frequency stimulation in hippocampal slices treated with cPAF and PAF-R antagonist BN52021 resulted in a small, long-lasting potentiation of EPSPs (1.29±0.05 relative to baseline from 40 to 50 min, n=10, P<0.05). EPSPs were not significantly potentiated in slices treated with a structurally-distinct PAF-R antagonist, CV-3988 (1.07±0.13 relative to baseline from 40 to 50 min, t=10, P=0.55). EPSP data from control slices and cPAF-exposed cells that did not bead are reproduced from FIG. 4d for comparison.

FIG. 9 shows the effect of mitochondrial calcium overload on synaptic fate following excitatory stimulation. High-frequency excitatory synaptic activity triggers post-synaptic calcium influx via NMDA receptors. Calcium is taken up from the cytosol by post-synaptic mitochondria. Under normal conditions (right) this causes a mild mitochondrial depolarization with low-level caspase activation, free radical production, and increased metabolic rates to power microtubule-mediated transport of proteins and organelles as well as post-synaptic actin stabilization. This delivers AMPA receptors and other proteins to the synapse, contributing to long-term potentiation. Elevated platelet-activating factor (PAF) concentrations (left) promote mitochondrial calcium overload, with mitochondrial swelling and severe depolarization. Local energy depletion, free radical production and caspase activation likely injure nearby microtubules, leading to dendritic beading as damaged proteins and organelles accumulate at the site of injury. Microtubule and actin injury both impair recruitment of new proteins to the synapse, causing failure of LTP and perhaps ultimately to weakened synaptic transmission. Chemical preconditioning appears to have its protective effect by re-directing the synaptic response toward LTP by preventing mitochondrial calcium overload and its toxic sequelae.

FIG. 10 shows that cPAF promotes mitochondrial depolarization following synaptic stimulation in hippocampal slices. a. Images of the net increase in rhodamine 123 fluorescence signal (with baseline fluorescence subtracted from post-stimulus images) demonstrate that in the presence of cPAF, portions of hippocampal area CAl distal to the Schaffer collateral stimulating electrode (arrows) show signs of mitochondrial depolarization in cPAF-treated slices that is greater than that in control slices following identical high-frequency synaptic stimulation (HFS). or, stratum oriens; pyr, pyramidal cell layer; rad, stratum radiatum. Scale bar, 80 μm. b. Quantitation of rhodamine 123 fluorescence in CA1 stratum radiatum during and following BFS shows a peak 5.90±1.86% increase in signal above baseline in the presence of cPAF (n=8 slices from 4 animals), and a smaller 1.37±0.46% increase in control slices (n=7 slices from 4 animals).

FIG. 11 shows that cPAF replaces long-term potentiation with dendritic beading in hippocampal slices. (a) Dendritic beading in a cPAF-exposed CAl pyramidal neuron 45 min after high-frequency Schaffer collateral stimulation (HFS), with no disruption of dendrite or spine morphology in following HFS in vehicle-treated cells. (b) HFS elicited dendritic beading in 11 of 19 cells from cPAF-treated slices, and in 0 of 13 cells from vehicle-treated slices (*P<0.001). PAF-R antagonists BN52021 and CV-3988 reduced dendritic beading to 1 of 10 and 1 of 7 cells, respectively (**P<0.05 vs. cPAF). (c) Excitatory synaptic transmission is strongly potentiated following HFS in vehicle-treated slices (2.66±0.44-fold relative to baseline at 40 to 50 min, n=13, P<0.001). In cPAF-treated slices, cells that did not develop dendritic beading underwent a smaller but significant potentiation (1.60±0.26 relative to baseline, n=8, P<0.05) while EPSPs in cells whose dendrites did bead were not potentiated at all (0.84±0.12 relative to baseline, n=11, P<0.01 vs. vehicle and P<0.05 vs. cPAF-treated cells without dendritic beading). Representative EPSPs from vehicle- (upper right) and beaded, cPAF-treated cells (lower right) are averages of 10 consecutive traces recorded at baseline and 50 min post-HFS. Scale bars 20 μm.

### DETAILED DESCRIPTION

The disclosed methods and compositions are readily understood by reference to the following detailed description of particular embodiments and the Example included therein and to the Figures and their previous and following description.

Provided are methods and compositions for treating or preventing HIV-related neurological disorders by administration of a compound having formula I. Thus, disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound of formula I is disclosed and discussed and a number of modifications that can be made to the compound are discussed, then each and every combination and permutation of the compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively disclosed. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

It must be noted that as used herein and in the appended claims, the singular forms “a”, “an”, and “the” include plural reference unless the context clearly dictates otherwise. Thus, for example, reference to “a molecule” includes a plurality of such molecules, reference to “the molecule” is a reference to one or more molecules and equivalents thereof known to those skilled in the art, and so forth.

Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein, and the material for which they are cited, are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Methods of Treating and Preventing HIV-1-Induced Neural Damage

A method of treating or preventing HIV-1 associated dendritic pathology in a brain cell is provided, comprising contacting the cell with a therapeutically effective dose of a compound of Formula I.

In one aspect, the disclosed compound can be a K channel modulator (e.g., a K_{ATP} channel agonist). Suitable compounds include benzothiadiazine derivatives such as those shown in Formula I.

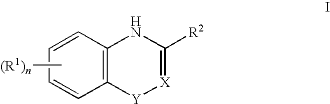

wherein
R¹ and R² can be, independent of one another, H, OH, NH₃, halogen, C₁₋₆ alkyl, C₁₋₆ alkoxyl, or NR³₂, where each R³ is, independent of the other, H, C₁₋₆ alkyl, or C₁₋₆ alkoxyl;
n can be 1-4;

### X can be N or CH; and

Y can be SO₂, O, C═O, CH₂, or NR³, where R³ can be H, C₁₋₆ alkyl, or C₁₋₆ alkoxyl.

Examples of suitable benzothiadiazine derivatives include, but are not limited to, diazoxide, 7-chloro-3-isopropylamino-4(1H)-1,2,4-benzothiadiazine-1,1-dioxide (see Lebrun et al., Diabetologia 2000, 43:723-732; Dupont et al., Z. Krystallogr. NCS 2005, 220), 6-fluoro-2-methylquinolin-4(1H)-one, or 6-chloro-2-methylquinolin-4(1H)-one (Becker et al, Br. J. Pharm. 2001, 134:375-385), including combinations thereof. In one particular example the compound is not diazoxide.

In one aspect, the disclosed compound can be a K channel modulator (e.g., a K_{ATP} channel agonist). Suitable compounds include nicotinic acid derivatives such as those shown in Formula II.

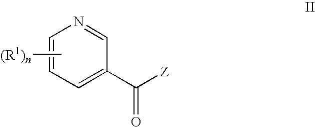

wherein

R¹ can be H, OH, NH₃, halogen, C₁₋₆ alkyl, C₁₋₆ alkoxyl, or NR³₂, where each R³ is, independent of the other, H, C₁₋₆ alkyl, or C₁₋₆ alkoxyl;

Z can be OH, NH₂, or NHR⁴, where R⁴ can be OH, NH₃, C₁₋₆ alkyl, C₁₋₆ alkoxyl, or C₂H₄R⁵, where R⁵ is OH, NH₃, NO₂, CN, halogen, Oalkyl, OC(O)CH₃, or furoxane (1,2,5-oxadiazole-2-oxide); and n can be 1-4.

Examples of suitable nicotinic acid derivatives include, but are not limited to, nicotinamide, nicorandil, and N-(2-(acetoxy)ethyl)-3-pyridinecarboxamide.

An example of a K_{ATP} channel agonist having the structure shown in Formula II is Nicorandil (N-[2-(2-nitrooxy)ethyl]-3-pyridinecarboxamide). The long-term use of nicorandil is described in Schalla et al., Long-term oral treatment with nicorandil prevents the progression of left ventricular hypertrophy and preserves viability, J Cardiovasc Pharmacol. 2005 April; 45(4):333-40, which is incorporated herein by reference for its teaching of long term use. Other references that describe the structure and administration of nicorandil are described in the literature (Nishikawa et al., Nicorandil regulates Bcl-2 family proteins and protects cardiac myocytes against hypoxia-induced apoptosis, J Mol Cell Cardiol. 2006 April; 40(4):510-9. Epub 2006 Mar. 9. Erratum in: J Mol Cell Cardiol. 2006 August; 41(2):371-2; Das and Sarkar, Is the sarcolemmal or mitochondrial K(ATP) channel activation important in the antiarrhythmic and cardioprotective effects during acute ischemia/reperfusion in the intact anesthetized rabbit model? Life Sci. 2005 Jul. 29; 77(11):1226-48; Miura and Miki, ATP-sensitive K+ channel openers: old drugs with new clinical benefits for the heart, Curr Vasc Pharmacol. 2003 October; 1(3):251-8; Khaliulin et al., Preconditioning improves postischemic mitochondrial function and diminishes oxidation of mitochondrial proteins, Free Radic Biol Med. 2004 Jul. 1; 37(1):1-9; and Harada et al. NO donor-activated PKC-delta plays a pivotal role in ischemic myocardial protection through accelerated opening of mitochondrial K-ATP channels, J Cardiovasc Pharmacol. 2004 July; 44(1):35-41, all of which are incorporated herein by reference for their teaching of the nature and administration of nicorandil).

Further examples of suitable compounds that can be used in the compositions and methods disclosed herein include, but are not limited to, alseroxylon, amlodipine, aprikalim, artilide fumarate, atenolol, benazepril hydrochloride, bimakalim, brotizolam, captopril, chromakalim, cinolazepam, clonazepam, clonidine hydrochloride, romakalim, deserpidine, diazixide, diltiazem, diltiazem hydrochloride, doxefazepam, emakalim, enalapril maleate, enalaprilat, estazolam, felodipine, flunitrazepam, flupirtine, guanethidine monosulfate, haloxazolam, hydralazine (apresoline), ibutilide fumarate, isoflurane, isradipine, lemakalim, levcromakalim, loprazolam, lorazepam, lormetazepam, metoprolol tartarate, midazolam, minoxidil, nicardipine, nicorandil, nifedipine, nimetazepam, nisoldipine, nitrendipine, nitroprusside (nipride), oxpenolol hydrochloride, oxyprenolol, pargyline hydrochloride, phenoxybenzamine, phentolamine, pinacidil, propafenone, propanolol, rauwolfia serpentina, rescinnamine, reserpine, rilmakalim, rilmazafone, sildenafil, sodium nitroprusside, spiroxazone, sulfonylurean, temazepam, tolazoline, trimethaphan, verapamil, PCO-400 (J. Vasc. Res., 1999, 36(6):516-523), 2-[2“(1”,3″-dioxolone)-2-methyl]-4-(2′-oxo-1′-pyrrolidinyl)-6-nitro-2H-1-benzopyran), 9-chloro-7-(2-chlorophenyl)-5H-pyrimido(5,4,-d) (2)-benzazepine, Ribi, CPG-11952, CGS-9896, CGP 42500, ZD-6169, P1075, P1060, Bay X 9227, Bay X 9228, WAY-120,491, WAY-120,129, Ro 31-6930, SR 44869, BRL 38226, S 0121, SR 46142A, SR 44994, BMS-191095, BMS-180448, EMD 60480, and MCC-134, including mixtures thereof.

A method of treating or preventing HIV-1 associated dendritic pathology in a brain cell is provided, comprising contacting the cell with a therapeutically effective dose of a mitochondrial ATP-sensitive potassium channel agonist (K⁺ ATP channel agonist), e.g., a compound of Formula I. In one aspect of a disclosed method of treating or preventing HIV-1 associated dendritic pathology in a brain cell, the mitochondrial ATP-sensitive potassium channel agonist is not diazoxide. In one aspect of a disclosed method of treating or preventing HIV-1 associated dendritic pathology in a brain cell, the mitochondrial ATP-sensitive potassium channel agonist is not Minoxidil. In one aspect of a disclosed method of treating or preventing HIV-1 associated dendritic pathology in a brain cell, the mitochondrial ATP-sensitive potassium channel agonist is not adenosine. In one aspect of a disclosed method of treating or preventing HIV-1 associated dendritic pathology in a brain cell, the mitochondrial ATP-sensitive potassium channel agonist is not s-nitoroso-N-acetylpenicillamine. In one aspect of a disclosed method of treating or preventing HIV-1 associated dendritic pathology in a brain cell, the mitochondrial ATP-sensitive potassium channel agonist is not BMS-191095. In one aspect of a disclosed method of treating or preventing HIV-1 associated dendritic pathology in a brain cell, the mitochondrial ATP-sensitive potassium channel agonist is not a combination of Adenosine+diazoxide+s-nitroso-N-acetylpenicillamine.

A method of treating or preventing HIV-1 associated dendritic pathology in a brain cell is provided, comprising contacting the cell with a therapeutically effective dose of a mitochondrial ATP-sensitive potassium channel agonist (K⁺ ATP channel agonist), e.g., a compound of Formula II. In one aspect of the disclosed method the compound of formula II is nicorandil.

Provided herein is a method of protecting a neuron from dysfunction induced by HIV-1 induced neurotoxicity comprising contacting the cell with a compound of Formula I.

Further provided is a method of treating or preventing HIV-1 associated dementia (HAD) in a subject in need of such treatment or prevention, comprising administering to the subject a therapeutically effective dose of a compound of Formula I.

A method of treating or preventing HIV-1 associated dendritic pathology in a brain cell is also provided, comprising contacting the cell with a therapeutically effective dose of an inhibitor of succinate dehydrogenase, e.g., a compound of Formula I. In one aspect of a disclosed method of treating or preventing HIV-1 associated dendritic pathology in a brain cell, the inhibitor of succinate dehydrogenase is not diazoxide. In one aspect of a disclosed method of treating or preventing HIV-1 associated dendritic pathology in a brain cell, the inhibitor of succinate dehydrogenase is not Minoxidil. In one aspect of a disclosed method of treating or preventing HIV-1 associated dendritic pathology in a brain cell, the inhibitor of succinate dehydrogenase is not adenosine. In one aspect of a disclosed method of treating or preventing HIV-1 associated dendritic pathology in a brain cell, the inhibitor of succinate dehydrogenase is not s-nitoroso-N-acetylpenicillamine. In one aspect of a disclosed method of treating or preventing HIV-1 associated dendritic pathology in a brain cell, inhibitor of succinate dehydrogenase is not BMS-191095. In one aspect of a disclosed method of treating or preventing HIV-1 associated dendritic pathology in a brain cell, the inhibitor of succinate dehydrogenase is not a combination of Adenosine+diazoxide+s-nitroso-N-acetylpenicillamine.

Further provided is a method of treating or preventing HIV-1 associated dendritic pathology in a brain cell, comprising contacting the cell with a therapeutically effective dose of a stimulator of production of reactive oxygen species, e.g., a compound of Formula I. In one aspect of a disclosed method of treating or preventing HIV-1 associated dendritic pathology in a brain cell, the stimulator of production of reactive oxygen species is not diazoxide. In one aspect of a disclosed method of treating or preventing HUV-1 associated dendritic pathology in a brain cell, the stimulator of production of reactive oxygen species is not Minoxidil. In one aspect of a disclosed method of treating or preventing HIV-1 associated dendritic pathology in a brain cell, the stimulator of production of reactive oxygen species is not adenosine. In one aspect of a disclosed method of treating or preventing HIV-1 associated dendritic pathology in a brain cell, the stimulator of production of reactive oxygen species is not s-nitoroso-N-acetylpenicillamine. In one aspect of a disclosed method of treating or preventing HIV-I associated dendritic pathology in a brain cell, the stimulator of production of reactive oxygen species is not BMS-191095. In one aspect of a disclosed method of treating or preventing HIV-1 associated dendritic pathology in a brain cell, the stimulator of production of reactive oxygen species is not a combination of Adenosine+diazoxide+s-nitroso-N-acetylpenicillamine.

Further provided is a method of treating or preventing HIV-1 associated dendritic pathology in a brain cell, comprising contacting the cell with a therapeutically effective dose of a composition comprising a K⁺ ATP channel agonist, e.g., a compound of Formula I and a compound selected from the group consisting of a modulator of adenosine receptor signaling and a molecule that inhibits mitochondrial hyperpolarization in a neural cell.

Further provided is a method of treating or preventing HIV-1 associated dendritic pathology in a brain cell, comprising contacting the cell with a therapeutically effective dose of a composition comprising an inhibitor of succinate dehydrogenase, e.g., a compound of Formula I and a compound selected from the group consisting of a modulator of adenosine receptor signaling and an inhibitor of mitochondrial hyperpolarization in a neural cell.

Further provided is a method of treating or preventing HIV-1 associated dendritic pathology in a brain cell, comprising contacting the cell with a therapeutically effective dose of a composition comprising a stimulator of production of reactive oxygen species, e.g., a compound of Formula I and a compound selected from the group consisting of a modulator of adenosine receptor signaling and an inhibitor mitochondrial hyperpolarization in a neural cell.

Further provided is a method of treating or preventing HIV-1 associated dendritic pathology in a brain cell, comprising contacting the cell with a therapeutically effective dose of a composition comprising at least two compositions selected from the group consisting of an inhibitor of succinate dehydrogenase, a stimulator of production of reactive oxygen species, a modulator of adenosine receptor signaling and an inhibitor of mitochondrial hyperpolarization in a neural cell.

HIV associated dementia (HAD) is comprised of a spectrum of conditions from the mild HIV-1 minor cognitive-motor disorder (MCMD) to severe and debilitating AIDS dementia complex. Symptoms begin with motor slowing and may progress to severe loss of cognitive function, loss of bladder and bowel control, and paraparesis. A classification system has been formulated for HIV associated dementia, wherein subjects are classified as being Stage 0 (Normal), Stage 0.5 (Subclinical or Equivocal), Stage 1 (Mild), Stage 2 (Moderate), Stage 3 (Severe), or Stage 4 (End-Stage).Thus, the subject of the provided method can therefore be classified as Stage O, Stage 0.5, Stage 1, Stage 2, Stage 3, or Stage 4.

By “treat” or “treatment” is meant a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for treatment of HAD is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. For example, in the case of HAD, to treat HAD in a subject can comprise improving the disease classification. (e.g. from stage 3 to stage 2, from stage 2 to stage 1, from stage 1 to 0.5 or from stage 0.5 to 0).

As used throughout, “preventing” means to preclude, avert, obviate, forestall, stop, or hinder something from happening, especially by advance planning or action. For example, to prevent HAD in a subject is to stop or hinder the subject from advancing in disease classification (e.g. from stage 0 to stage 0.5, from stage 0.5 to stage 1, from stage 1 to stage 2, from stage 2 to stage 3, or from stage 3 to stage 4). The timing and frequency of administration of agents in order to distinguish between the activation of mitochondrial K-ATP channels during or before neurodegeneration can be determined in in vivo models of neurodegeneration.

Microglia, macrophages and astrocytes are major HIV-1 targets in the brain, whereas HIV-1 infected neurons have been rarely observed. This indicates that indirect mechanisms may account for the severe neuronal damage observed in these patients. In addition to the production of cytokines, HIV-1 infected and/or functionally activated mononuclear cells and astrocytes can produce a number of soluble mediators, including the structural and regulatory proteins gp120, Tat, and platelet activating factor (PAF), which can exert damaging effects on both developing and mature neural tissues.

The disclosed method can be further combined with other therapeutic approaches for the treatment of HIV-1 infection or HAD. Thus, the disclosed method can further comprise administering to the subject an antiretroviral compound. Antiretroviral drugs inhibit the reproduction of retroviruses such as HIV. Antiretroviral agents are virustatic agents which block steps in the replication of the virus. The drugs are not curative; however continued use of drugs, particularly in multi-drug regimens, can significantly slow disease progression. There are three main types of antiretroviral drugs, although only two steps in the viral replication process are blocked. Nucleoside analogs, or nucleoside reverse transcriptase inhibitors (NRTIs), act by inhibiting the enzyme reverse transcriptase. Because a retrovirus is composed of RNA, the virus must make a DNA strand in order to replicate itself. Reverse transcriptase is an enzyme that is essential to making the DNA copy. The nucleoside reverse transcriptase inhibitors are incorporated into the DNA strand. This is a faulty DNA molecule that is incapable of reproducing. The non-nucleoside reverse transcriptase inhibitors (NNRTIs) act by binding directly to the reverse transcriptase molecule, inhibiting its activity. Protease inhibitors act on the enzyme protease, which is essential for the virus to break down the proteins in infected cells. Without this essential step, the virus produces immature copies of itself, which are non-infectious. A fourth class of drugs called fusion inhibitors block HIV from fusing with healthy cells.

Thus, the antiretroviral compound can comprise one or more molecules selected from the group consisting of protease inhibitors [PI], fusion inhibitors, nucleoside reverse transcriptase inhibitors [NRTI], and non-nucleoside reverse transcriptase inhibitors [NNRTI].

Thus, the antiretroviral compound of the provided method can be a PI, such as a PI selected from the group consisting of Indinavir, Amprenavir, Nelfinavir, Saquinavir, Fosamprenavir, Lopinavir, Ritonavir, and Atazanavir, or any combinations thereof.

Thus, the antiretroviral compound of the provided method can be a fusion inhibitor, such as for example Enfuvirtide.

Thus, the antiretroviral compound of the provided method can be a NRTI, such as a NRTI selected from the group consisting of Abacavir, Stavudine, Didanosine, Lamivudine, Zidovudine, Zalcitabine, Tenofovir, and Emtricitabine, or any combinations thereof.

Thus, the antiretroviral compound of the provided method can be a NNRTI, such as a NNRTI selected from the group consisting of Efavirenz, Nevirapine, and Delavirdine.

The disclosed method can further comprise administering to the subject an inhibitor of mitochondrial hyperpolarization. As used herein, mitochondrial hyperpolarization (MIP) refers to an elevation in the mitochondrial transmembrane potential, ΔΨₘ (delta psi), i.e., negative inside and positive outside). The ΔΨₘ is the result of an electrochemical gradient maintained by two transport systems—the electron transport chain and the F₀F₁-ATPase complex. For a review, see Perl et al. 2004 Trends in Immunol. 25:360-367. Briefly, the electron transport chain catalyzes the flow of electrons from NADH to molecular oxygen and the translocation of protons across the inner mitochondrial membrane, thus creating a voltage gradient with negative charges inside the mitochondrial matrix. F₀F₁-ATPase utilizes the extruded proton to synthesize ATP. MHP leads to uncoupling of oxidative phosphorylation, which disrupts ΔΨₘ and damages integrity of the inner mitochondrial membrane. Disruption of ΔΨₘ has been proposed as the point of no return in cell death signaling. This releases cytochrome c and other cell-death-inducing factors from mitochondria into the cytosol. Thus, the inhibitor of the present method can be a F₀F₁-ATPase agonists.

The inhibitor of the present method can be an electron transport inhibitor. The electron transport chain (ETC) is the biomolecular machinery present in mitochondria that couples the flow of electrons to proton pumps in order to convert energy from sugar to ATP. The electron transport chain couples the transfer of an electron from NADH (nicotinamide adenine dinucleotide) to molecular oxygen (O₂) with the pumping of protons (H⁺) across a membrane. The charge gradient that results across the membrane serves as a battery to drive ATP Synthase. The electron transport chain is made up of several integral membrane complexes: NADH dehydrogenase (complex I), Coenzyme Q—cytochrome c reductase (complex III), and Cytochrome c oxidase (complex IV). Succinate—Coenzyme Q reductase (Complex II) connects the Krebs cycle directly to the electron transport chain.

Thus, the inhibitor of the provided method can be an inhibitor of any component of the ETC. Thus, the inhibitor can be an inhibitor of complex I, II, III, or IV. For example, diphenylene iodonium (DPI) and rotenone are specific inhibitors of complex I, succinate-q reductase (TTFA) is an inhibitor of complex II, antimycin A and myxothiazole are inhibitors of complex III, and potassium cyanide (KCN) is an inhibitor of complex IV. Thus, the inhibitor of the provided method can be selected from the group consisting of diphenylene iodonium (DPI), rotenone, antimycin, myxothiazole, succinate-q reductase (TTFA), and potassium cyanide (KCN).

The inhibitor of the present method can be an uncoupler. As used herein an “uncoupler” is a substance that allows oxidation in mitochondria to proceed without the usual concomitant phosphorylation to produce ATP; these substances thus “uncouple” oxidation and phosphorylation. As an example, Trifluorocarbonylcyanide Phenylhydrazone (FCCP) is a chemical uncoupler of electron transport and oxidative phosphorylation. FCCP permeabilizes the inner mitochondrial membrane to protons, destroying the proton gradient and, in doing so, uncouples the electron transport system from the oxidative phosphorylation system. In this situation, electrons continue to pass through the electron transport system and reduce oxygen to water, but ATP is not synthesized in the process.

The uncoupler of the present method can agonize, antagonize or modulate the expression of endogenous mitochondrial uncoupling proteins (UCPs). As a non-limiting example, the uncoupler of the present method can be the beta-adrenergic agonist CL-316,243 (disodium (R,R)-5-(2-((2-(3-chlorophenyl)-2-hydroxyethyl)-amino)propyl)-1,3-benzodioxole-2,3-dicarboxylate) (Yoshida et. al., Am J. Physiol. 1998. 274(3 Pt 1): p. E469-75).

The uncoupler of the present method can be a protonophore. Thus, the inhibitor of the present method can be a protonophore. As used herein, a “protonophore” is a molecule that allows protons to cross lipid bilayers. The protonophore can be FCCP. The protonophore can also be 2,4,-dinitrophenol (DNP). The protonophore can be also m-chlorophenylhydrazone (CCCP). The protonophore can also be pentachlorophenol (PCP).

The disclosed method can further comprise contacting the cell with an antioxidant. Generally, antioxidants are compounds that react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The herein disclosed antioxidant can be any antioxidant, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly α-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavonoids and flavonoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

Thus, the disclosed method can further comprise contacting the cell with an antioxidant selected from the group consisting of tauroursodeoxycholic acid (TUDCA), N-acetylcysteine (NAC) (600-800 mg/day), Mito-Coenzyme Q10 (Mito-CoQ) (300-400 mg/day), Mito-VitaminE (Mito-E) (100-1000 mg/day), Coenzyme Q10 (300-400 mg/day), and idebenone (60-120 mg/day).

N-acetylcysteine (NAC) is used to replenish Glutathione (GSH) that has been depleted in HIV-infected individuals by acetaminophen overdose. (De Rosa S C, Zaretsky M D, Dubs J G, Roederer M, Anderson M, Green A, Mitra D, Watanabe N, Nakamura H, Tjioe I, Deresinski S C, Moore W A, Ela S W, Parks D, Herzenberg L A, Herzenberg L A. N-acetylcysteine replenishes glutathione in HIV infection. European Journal of Clinical Investigation, 30(10):915). Thus, in one embodiment of the provided invention, NAC is not used to replenish Glutadione (GSH) in HIV-infected subjects. In another embodiment of the method NAC is not used to treat HAD.

Coenzyme Q10 has been used to treat patients having the AIDS related complex. (Folkers K, Hanioka T, Xia L J, McRee J T Jr, Langsjoen P. Coenzyme Q10 increases T4/T8 ratios of lymphocytes in ordinary subjects and relevance to patients having the AIDS related complex. Biochem Biophys Res Commun. 1991 Apr. 30; 176(2):786-91.)

Bile acids such as TUDCA lead to a significant improvement in serum transaminase activities in subjects with hepatitis B and C. (Chen W, Liu J, Gluud C. Bile acids for viral hepatitis. Cochrane Database Syst Rev. 2003; (2):CD003181.) Thus, in one embodiment of the provided invention, Coenzyme Q10 is not used to treat patients having the AIDS related complex. In another embodiment of the method Coenzyme Q10 is not used to treat HAD.

Idebenone has been used to treat subjects with senile cognitive decline (Bergamasco B, Villardita C, Coppi R. Effects of idebenone in elderly subjects with cognitive decline. Results of a multicentre clinical trial. Arch Gerontol Geriatr. 1992 November-December; 15(3):279-86.) Thus, in one embodiment of the provided invention, Idebenone not used to treat subjects with senile cognitive decline. In another embodiment of the method Idebenone is not used to treat HAD.

The disclosed method can further comprise administering to the subject a neurotoxin inhibitor. The inhibitor can be a TNFα inhibitor, including TNFα-inhibitory monoclonal antibodies (e.g., etanercept), phosphodiesterase (PDE)-4 inhibitors (such as IC485, which can reduce TNFα production), thalidomide and other agents.

Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept has been evaluated in HIV-infected subjects receiving highly active antiretroviral therapy (HAART) (Sha B E, Valdez H, Gelman R S, Landay A L, Agosti J, Mitsuyasu R, Pollard R B, Mildvan D, Namkung A, Ogata-Arakaki D M, Fox L, Estep S, Erice A, Kilgo P, Walker R^{E}, Bancroft L, Lederman M M. Effect of etanercept (Enbrel) on interleukin 6, tumor necrosis factor alpha, and markers of immune activation in HIV-infected subjects receiving interleukin 2. AIDS Res Hum Retroviruses. 2002 Jun. 10; 18(9):661-5).

IC485 is an orally administered, small molecule inhibitor of PDE4. Inhibition of PDE4 leads to an increase in the second messenger, cAMP, within cells. This inhibition may in turn reduce the cell's production of tumor necrosis factor alpha (TNF-alpha) and a variety of other inflammatory mediators. IC485 is being evaluated in patients with chronic obstructive pulmonary disease.

The inhibitor can be a PAF receptor antagonist (such as lexipafant, WEB2086, WEB2170, BN-52021 or PMS-601), a PAF degrading-enzyme such as PAF-acetylhydrolase (PAF-AH), or a molecule that regulates the expression of PAF-AH (such as pioglitazone and other PPAR-gamma inhibitors).

Lexipafant has been used improve cognitive dysfunction in HIV-infected people (Schifitto G, Sacktor N, Marder K, McDermott M P, McArthur J C, Kieburtz K, Small S, Epstein L G. Randomized trial of the platelet-activating factor antagonist lexipafant in HIV-associated cognitive impairment. Neurological AIDS Research Consortium. Neurology. 1999 Jul. 22; 53(2):391-6). Lexipafant can be administered at for example 500 mg/day.

PMS-601 inhibits proinflainmatory cytokine synthesis and HIV replication (Martin M, Serradji N, Dereuddre-Bosquet N, Le Pavec G, Fichet G, Lamouri A, Heymans F, Godfroid J J, Clayette P, Dormont D. PMS-601, a new platelet-activating factor receptor antagonist that inhibits human immunodeficiency virus replication and potentiates zidovudine activity in macrophages. Antimicrob Agents Chemother. 2000 November; 44(11):3150-4.)

TNF-alpha-mediated neuronal apoptosis can also be blocked by co-incubation with PAF acetylhydrolase (PAF-AH) (Perry S W, Hamilton J A, Tjoelker L W, Dbaibo G, Dzenko K A, Epstein L G, Hannun Y, Whittaker J S, Dewhurst S, Gelbard H A. Platelet-activating factor receptor activation. An initiator step in HIV-1 neuropathogenesis. J Biol. Chem. 1998 Jul. 10; 273(28):17660-4).

Pioglitazone can inhibit PAF-induced morphological changes through PAF-AH (Sumita C, Maeda M, Fujio Y, Kim J, Fujitsu J, Kasayama S, Yamamoto I, Azuma J. Pioglitazone induces plasma platelet activating factor-acetylhydrolase and inhibits platelet activating factor-mediated cytoskeletal reorganization in macrophage. Biochim Biophys Acta. 2004 Aug. 4; 1673(3):115-21).

Phosphatidylcholines (1-O-alcoxy-2-amino-2-desoxy-phosphocholines and 1-pyrene-labeled analogs) were synthesized and used to examine interactions with recombinant human PAF-AH (Deigner B P, Kinscherf R, Claus R, Fyrnys B, Blencowe C, Hermetter A. Novel reversible, irreversible and fluorescent inhibitors of platelet-activating factor acetylhydrolase as mechanistic probes. Atherosclerosis. 1999 May; 144(1):79-90).

The disclosed method can further comprise administering to the subject an inhibitor of GSK-3β. The inhibitor can be valproate or lithium.

Valproate has been administered to HIV-infected patients receiving efavirenz or lopinavir (DiCenzo R, Peterson D, Cruttenden K, Morse G, Riggs G, Gelbard H, Schifitto G. Effects of valproic acid coadministration on plasma efavirenz and lopinavir concentrations in human immunodeficiency virus-infected adults. Antimicrob Agents Chemother. 2004 November; 48(11):4328-31). A typical dose of valproate comprises 250 mg twice daily.

The disclosed method can further comprise administering to the subject a compound that enhances CNS uptake. Ritonavir influences levels of coadministered drugs in the CNS, due to effects on the activity of drug transporters located at the BBB (Haas D W, Johnson B, Nicotera J, Bailey V L, Harris V L, Bowles F B, Raffanti S, Schranz J, Finn T S, Saah A J, Stone J Effects of ritonavir on indinavir pharmacokinetics in cerebrospinal fluid and plasma Antimicrob Agents Chemother. 2003 July; 47(7):2131-7).

The disclosed methods can further comprise administering a drug that inhibits the P-glycoprotein drug efflux pump, or multidrug resistance-associated proteins at the blood-brain-barrier (BBB). These include LY-335979 (Choo E F, Leake B, Wandel C, Imamura H, Wood A J, Wilkinson G R, Kim R B. Pharmacological inhibition of P-glycoprotein transport enhances the distribution of HIV-1 protease inhibitors into brain and testes. Drug Metab Dispos. 2000 June; 28(6):655-60) and PSC-833 and GF120918 (Pgp blockers) (Polli J W, Jarrett J L, Studenberg S D, Humphreys J E, Dennis S W, Brouwer K R, Woolley J L. Role of P-glycoprotein on the CNS disposition of amprenavir (141W94), an HIV protease inhibitor. Pharm Res. 1999 August; 16(8):1206-12; Kemper E M, van Zandbergen A E, Cleypool C, Mos H A, Boogerd W, Beijnen J H, van Tellingen O. Increased penetration of paclitaxel into the brain by inhibition of P-Glycoprotein. Clin Cancer Res. 2003 July; 9(7):2849-55) as well as MK571 (a specific Mrp family inhibitor):

The disclosed method can further comprise administering to the subject a microglial deactivator. Minocyclin is a potent microglial deactivator (Wu D C, Jackson-Lewis V, Vila M, Tieu K, Teismann P, Vadseth C, Choi D K, Ischiropoulos H, Przedborski S. Blockade of microglial activation is neuroprotective in the 1-methyl-4-phenyl-1,2,3,6-tetrahydropyridine mouse model of Parkinson disease. J. Neurosci. 2002 Mar. 1; 22(5):1763-71; Yrjanheikki J, Keinanen R, Pellikka M, Hokfelt T, Koistinaho J. Tetracyclines inhibit microglial activation and are neuroprotective in global brain ischemia. Proc Natl Acad Sci USA. 1998 Dec. 22; 95(26):15769-74). Further, minocycline can potently inhibit HIV-1 viral production from microglia (Si Q, Cosenza M, Kim M O, Zhao M L, Brownlee M, Goldstein H, Lee S. A novel action of minocycline: inhibition of human immunodeficiency virus type 1 infection in microglia. J. Neurovirol. 2004 October; 10(5):284-92). Thus, the microglial deactivator can be minocycline. A typical dosage of minocyclin comprises 200 mg/day.

Other microglial deactivators that can be used in the present methods include PDE4 inhibitors (described above).

The disclosed method can further comprise administering to the subject an inhibitor of glutamate damage. The inhibitor can be a beta-lactam antibiotic such as for example ceftriaxone, which can have direct effects on glutamate transporter expression.

When delivered to animals, the beta-lactam ceftriaxone increases both brain expression of GLT1 that inactivates synaptic glutamate (Rothstein J D, Patel S, Regan M R, Haenggeli C, Huang Y H, Bergles D E, Jin L, Dykes Hoberg M, Vidensky S, Chung D S, Toan S V, Bruijn L I, Su Z Z, Gupta P, Fisher P B. Beta-lactam antibiotics offer neuroprotection by increasing glutamate transporter expression. Nature. 2005 Jan. 6; 433(7021):73-7) A typical dosage of cephtriaxone is 50 mg/kg/day.

A dose-dependent inhibition of high affinity glutamate uptake sites is observed after addition of exogenous recombinant human TNFα to human fetal astrocytes (PHFAs) (Fine S M, Angel R A, Perry S W, Epstein L G, Rothstein J D, Dewhurst S, Gelbard H A. Tumor necrosis factor alpha inhibits glutamate uptake by primary human astrocytes. Implications for pathogenesis of HIV-1 dementia. J Biol. Chem. 1996 Jun. 28; 271(26):15303-6). Thus, the inhibitor of glutamate damage can be a TNFα inhibitor or a microglial deactivator (descrived above), which can have indirect effects on glutamate transporters.

Compositions

Further provided is a composition, comprising a K⁺ ATP channel agonist and a compound selected from the group consisting of a modulator of adenosine receptor signaling and a molecule that inhibits mitochondrial hyperpolarization in a neural cell. In on aspect, the composition comprising a K⁺ ATP channel agonist, the agonist can be a compound of Formula I. In an example of a composition comprising the compound of Formula I, the compound is diazoxide. In an example of a composition comprising the compound of Formula I, the compound is 7-chloro-3-isopropylamino-4(1H)-1,2,4-benzothiadiazine-1,1-dioxide. In a ifurther example of a composition comprising the compound of Formula I, the compound is 6-fluoro-2-methylquinolin-4(1H)-one. In a further example of a composition comprising the compound of Formula I, the compound is 6-chloro-2-methylquinolin-4(1H)-one. In on aspect, the composition comprising a K⁺ ATP channel agonist, the agonist can be a compound of Formula II. In an example of a composition comprising the compound of Formula II, the compound is nicorandil.

Thus, provided is a composition comprising nicorandil and a compound selected from the group consisting of a modulator of adenosine receptor signaling and a molecule that inhibits mitochondrial hyperpolarization in a neural cell.

Further provided is a composition, comprising an inhibitor of succinate dehydrogenase and a compound selected from the group consisting of a modulator of adenosine receptor signaling and a molecule that inhibits mitochondrial hyperpolarization in a neural cell.

Further provided is a composition, comprising a stimulator of production of reactive oxygen species and a compound selected from the group consisting of a modulator of adenosine receptor signaling and a molecule that inhibits mitochondrial hyperpolarization in a neural cell.

Further provided is a composition, comprising at least two compositions selected from the group consisting of a K+ATP channel agonist, an inhibitor of succinate dehydrogenase, a stimulator of production of reactive oxygen species, a modulator of adenosine receptor signaling and an inhibitor of mitochondrial hyperpolarization in a neural cell.

Any of the compounds described herein can be the pharmaceutically-acceptable salt thereof. In one aspect, pharmaceutically-acceptable salts are prepared by treating the free acid with an appropriate amount of a pharmaceutically-acceptable base. For example, one or more hydrogen atoms of the SO₃H group can be removed with a base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

In another aspect, if the compound possesses a basic group, it can be protonated with an acid such as, for example, HCl or H₂SO₄, to produce the cationic salt. For example, the techniques disclosed in U.S. Pat. No. 5,436,229 for producing the sulfate salts of argininal aldehydes, which is incorporated by reference in its entirety, can be used herein. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

It is contemplated that the pharmaceutically-acceptable salts of the compounds described herein can be used as prodrugs or precursors to the active compound prior to the administration. For example, if the active compound is unstable, it can be prepared as its salt form in order to increase stability in dry form (e.g., powder).

Therapeutic Doses

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, the disclosed K⁺ ATP channel agonists can be administered at published dosages, such as those approved for human use. For example, longer-term use of compounds of Formula I, e.g., diazoxide in subjects, using daily 5 mg/lcg intraperitoneal injections is effective. In a further example a compound of Formula II, e.g., nicorandil, can be administered to a subject as a solution having a 3 to 30 μM concentration. Nicorandil can be administered orally, e.g., in an 0.003% nicorandil-containing diet or orally for 5 consecutive days in a dose of 5 mg kg. Nicorandil can be give as a bolus of 0.003-1 mg/kg, for example, 3 micrograms/kg/min.

A typical daily dosage of the disclosed modulators of adenosine receptor signaling used alone can range from about 0.05 to 5 mg/kg of body weight or more per day, depending on the factors mentioned above. In one aspect, the disclosed A2AR antagonists (e.g. ATL455, KW6002 and ZM241685) can be administered at doses ranging from 0.3 to 3 mg/kg of body weight per day; KW6002 can be administered to humans at doses up to 40 mg/day. In another aspect, the disclosed A2AR agonists (e.g. ATL146e, ATL313 and CGS21680) can be administered at from 0.05 to 50 mg/kg of body weight per day.

A typical daily dosage of the disclosed inhibitors of hyperpolarization used alone can range from about 0.001 mg/kg to up to 50 mg/kg of body weight or more per day, depending on the factors mentioned above.

In another aspect, the disclosed inhibitors of the ECC (e.g., DPI, rotenone, antimycin, myxothiazole, TTFA, and KCN can be administered at from 0.001 mg/kg to 1 mg/kg of body weight per day. In another aspect, the disclosed protonophore (e.g., FCCP, DNP, CCCP, PCP) can be administered at from 0.001 mg/kg to 1 mg/kg of body weight per day. In one aspect, the disclosed beta-adrenergic agonist CL-316,243 can be administered at 0.01 to up to 1 mg/kg, including 0.1 mg/kg, of body weight or more per day.

In another aspect, the disclosed antioxidants can be administered at from 1 mg/day to 1000 mg/day. As non-limiting examples, N-acetylcysteine (NAC) can be administered at from about 600 mg/day to 800 mg/day; Mito-Coenzyme Q10 (Mito-CoQ) can be administered at from about 300 mg/day to 400 mg/day; Mito-VitaminE (Mito-E) can be administered from about 100 to 1000 mg/day); Coenzyme Q10 can be administered from about 300 mg/day to 400 mg/day; and idebenone can be administered at from about 60 mg/day to 120 mg/day.

Pharmaceutically Acceptable Carriers

The compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered intracranially intravenously, intraperitoneally, intramuscularly, subcutaneously, intracavity, or transdermally.

Some of the herein disclosed compositions are recognized to cross the blood-brain-barrier. For example, CGS21680 (Agnati L F, Leo G, Vergoni A V, Martinez E, Hockemeyer J, Lluis C, Franco R, Fuxe K, Ferre S, Neuroprotective effect of L-DOPA co-administered with the adenosine A2A receptor agonist CGS 21680 in an animal model of Parkinson's disease. Brain Res Bull. 2004 Aug. 30; 64(2):155-64); Istradefylline (Weiss S M, Benwell K, Cliffe I A, Gillespie R J, Knight A R, Lerpiniere J, Misra A, Pratt R M, Revell D, Upton R, Dourish C T. Discovery of nonxanthine adenosine A2A receptor antagonists for the treatment of Parkinson's disease. Neurology. 2003 Dec. 9; 61(11 Suppl 6):S101-6; Chase T N, Bibbiani F, Bara-Jimenez W, Dimitrova T, Oh-Lee J D. Translating A2A antagonist KW6002 from animal models to parkinsonian patients. Neurology. 2003 Dec. 9; 61(11 Suppl 6):S107-11); and ATL455 can cross the BBB.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The compositions can be administered orally or parenterally (e.g., intravenously, intramuscular injection, by intraperitoneal injection, transdermally, extracorporeally, intracranially, topically or the like, including topical intranasal administration or administration by inhalant. As used herein, “intracranial administration” means the direct delivery of substances to the brain including, for example, intrathecal, intracisternal, intraventricular or trans-sphenoidal delivery via catheter or needle. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Models of HAD

As shown herein, PAF can lower the threshold for synaptic injury, and by impairing synaptic function, beading can serve as an important functional marker of dendritic injury, and can underlie the reversible impairments of neuronal function seen in HAD. Equally important, the in vitro and in vivo models of dendritic injury disclosed herein are sensitive and reproducible, and have great utility to determine the ability of adjunctive therapies to restore function (i.e., synaptic transmission) during exposure to HIV-1 neurotoxins.

A method of screening for inhibitors of HIV-1 associated dendritic pathology in a brain cell is provided, comprising: a) contacting a hippocampal slice with the putative inhibitor compound; b) contacting the hippocampal slice of step a) with platelet-activating factor; c) stimulating the hippocampal slice of step b) with high frequency stimulation; and d) detecting a reduction in HIV-1 associated dendritic pathology in a cell in the hippocampal slice contacted with the putative inhibitor, a reduction in dendritic pathology, compared to dendritic morphology in the absence of the putative inhibitor, indicating that the compound is an inhibitor of dendritic pathology. In one aspect of the method for identifying inhibitors of HIV-associated dendritic pathology, the comparison may be made by reference to know dendritic morphology described in the literature. In another aspect of the method for identifying inhibitors of HIV-associated dendritic pathology, the comparison may be made by reference to a hippocampal slice that has been contacted by PAF, subjected to HFS and not contacted with the putativie inhibitor (e.g., a control).

Thus, provided is a model of HIV-1 associated dendritic pathology, comprising a) contacting a hippocampal slice with platelet-activating factor; and b) stimulating the hippocampal slice of a) with high frequency stimulation. This model can be used to study HAD.

The effect of HAD can be studied in this model by measuring long term potentiation (see examples). In addition to long-term potentiation, the effect of exposure to PAF and mK_{ATP} channel agonists on mitochondrial depolarization and generation of reactive oxygen species following high frequency stimulation can be measured.

In vivo modes of HAD include the SCID mouse model (see Example 3). The model mouse, exhibiting one or more symptoms or clinical measures of HAD, are contacted with mK_{ATP} channel agonist in vivo, e.g., intraperitoneally or intrathecally. Other model animals are not contacted with mK_{ATP} channel agonist as controls. The brain tissue of the treated and untreated animals is analyzed for indicial of HAD to confirm the efficacy of the mK_{ATP} channel agonist tested.

In vivo models of neurodegeneration are provided using, for example, animals that are transgenic for Alzheimer's disease gene products and varying dose and times of agents such as diazoxide, minoxidil, or diazoxide+adenosine+s-nitroso-N-acetylpenicillamine. The timing and frequency of administration of agents in order to activate mK_{ATP} channels during (as opposed to before) neurodegeneration can be optimized using the models disclosed herein and elsewhere.

The following examples are set forth below to illustrate the methods and results according to the present invention. These examples are not intended to be inclusive of all aspects of the present invention, but rather to illustrate representative methods and results. These examples are not intended to exclude equivalents and variations of the present invention which are apparent to one skilled in the art.

### Example 1

### Synaptic Activity Becomes Excitotoxic in Neurons Exposed to Elevated Levels of Platelet-Activating Factor

### Abstract

In hippocampal slices exposed to a stable platelet-activating factor analogue, tetanic stimulation that normally induces long-term synaptic potentiation instead promoted development of calcium- and caspase-dependent dendritic beading. Chemical preconditioning with diazoxide, a mitochondrial ATP-sensitive potassium channel agonist, prevented dendritic beading and restored long-term potentiation. In contrast to models invoking excessive glutamate release, these results indicate that physiologic synaptic activity triggers excitotoxic dendritic injury during chronic neuroinflammation. Furthermore, administration of compounds of Formula I represents a novel therapeutic strategy for preventing excitotoxic injury while preserving physiologic plasticity.

### Results

cPAF Exposure Leads to Dendritic Beading and Spine Loss

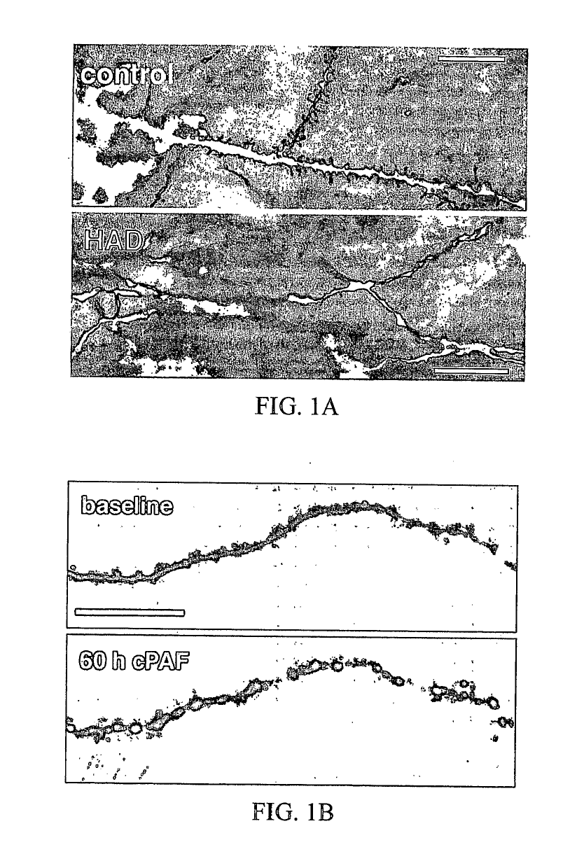

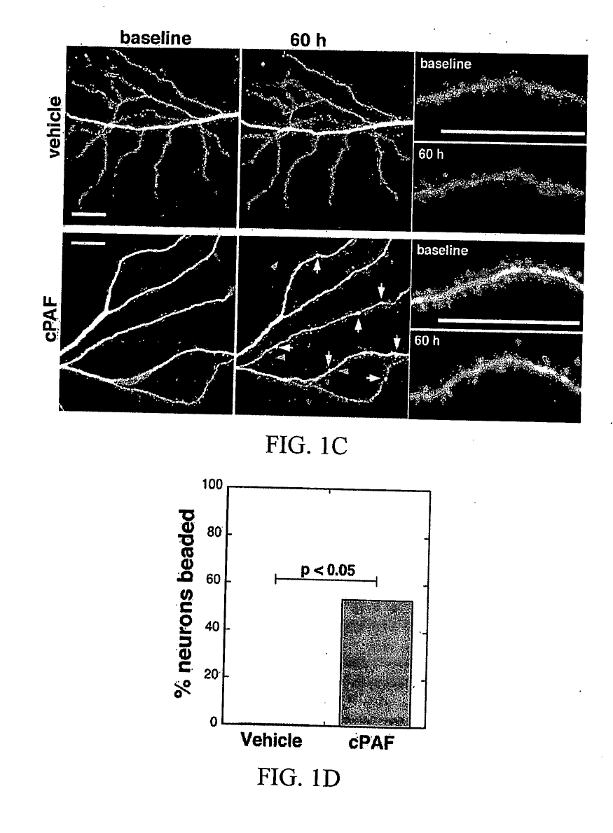

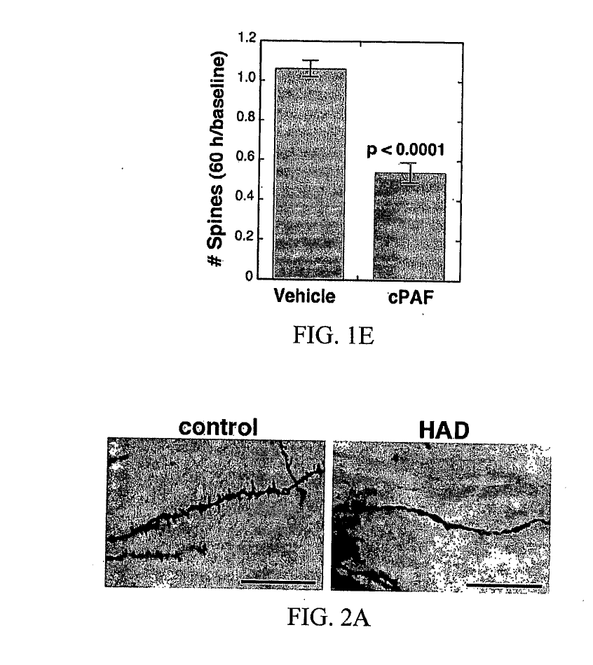

Because in vitro studies have implicated PAF as a common downstream mediator for the actions of diverse neurotoxins in HAD (24), whether exposure to elevated PAF signaling can recapitulate the dendritic injury seen in HAD was tested. Golgi-stained cortical neurons in tissue from patients with HAD (n=5) showed focal swellings, or beading, and very few spines, while those in tissue from HIV-1 seropositive patients without neurologic disease (n=5) had many spines and no beading (FIG. 1a). This is consistent with previous studies of HAD neuropathology (7).

Dendrites in dissociated hippocampal cultures developed nearly identical pathology during a 60-hour exposure to a sublethal (130 nM) dose of cPAF (FIG. 1b). Hippocampal neurons were studied in vitro because hippocampal dendrites are injured in HAD (6) and PAF effects on synaptic function and excitotoxicity have been well studied in these model systems. Neurons in 3-4 week old cultures were transfected with red or yellow fluorescent proteins (mRFP or EYFP) and compared images of the same cells taken before and after cPAF exposure. While dendritic arbors remained grossly intact, maintaining similar branching and projection patterns (FIG. 1c), cPAF exposure led to dendritic beading and loss of dendritic spines. Small focal swellings developed along dendritic shafts in 56% of serially-imaged cPAF-exposed cells, while no beading developed in control cells (FIG. 1d; n=17 cells from 4 cultures, P<0.05). Numbers of dendritic spines remained stable in control cells (6.2±4.1% increase over 6.0 hours), but decreased by 45.1±5.0% with cPAF exposure (FIG. 1e; n=10 cells from 4 cultures, P<0.0001). Most spines in these cultures were short and mushroom-shaped at baseline, but disruption of mature spines in cPAF-treated cells was often accompanied by the appearance of longer spines and filopodia (FIGS. 1b, c). 130 nM cPAF did not lead to death of any of the cells that were imaged, or decrease overall neuronal survival in cultures as assessed by Hoechst and propidium iodide staining (80.2±4.3% cPAF vs. 79.0±2.3% vehicle control, n=3 cultures, P=0.77).

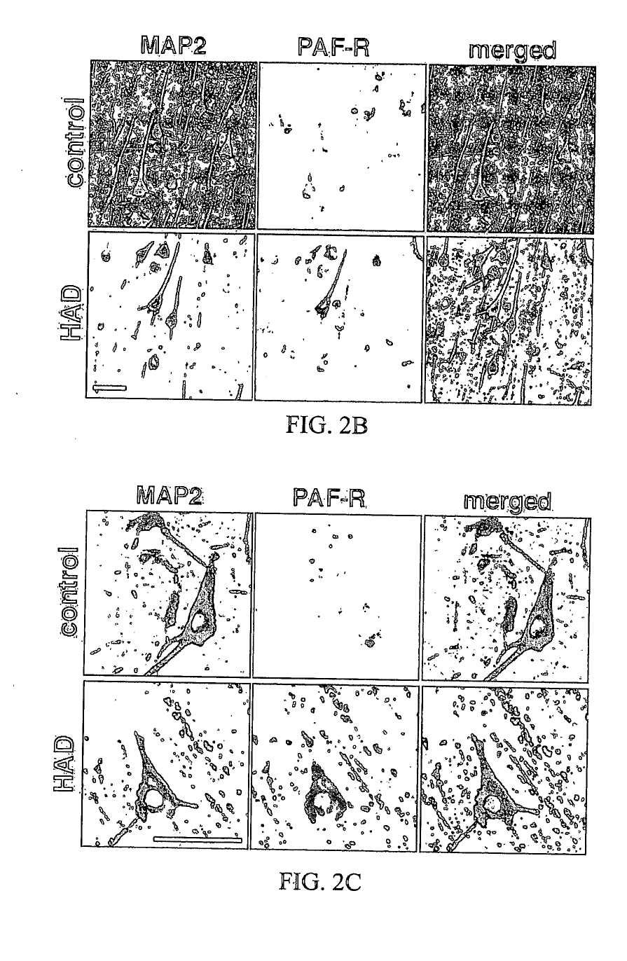

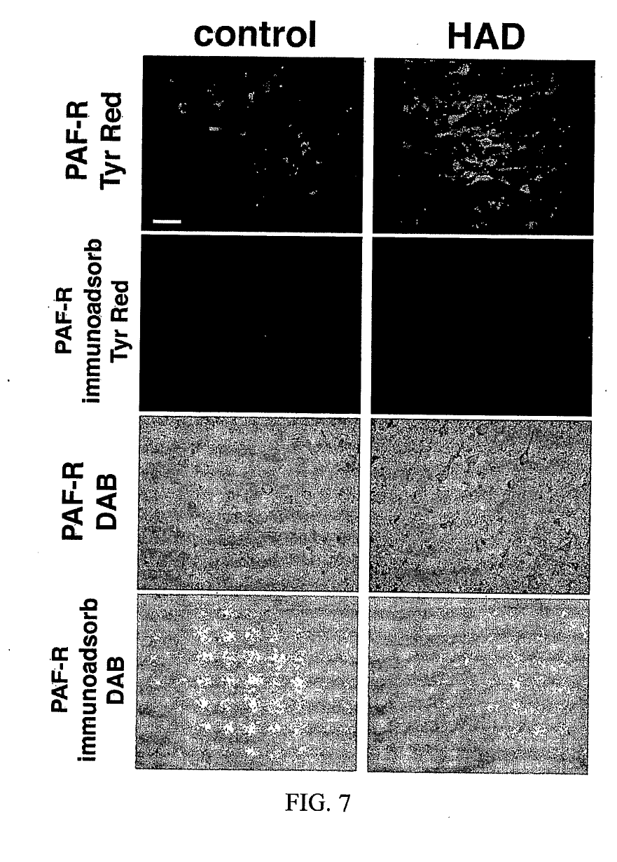

Immunohistochemical staining of cortical tissue from patients with HAD and dendritic injury (FIG. 2a) demonstrated increased expression of PAF receptor (PAF-R) on neuronal cell bodies and dendrites compared to HIV-1 seropositive controls (FIGS. 2b, c). Co-immunostaining for microtubule-associated protein 2 (MAP2), a marker for dendrites and neuronal cell bodies, showed PAF-R expression on nearly all dendrites in both HAD and control tissue. Non-neuronal cells expressing PAF-R are also present in both conditions, and PAF-R staining was eliminated by pre-incubation with a PAF-R-derived peptide antigen (FIG. 7), corroborating the specificity of PAF-R immunohistochemistry in this tissue. Wile MAP2-positive dendrites were markedly reduced in HAD, PAF-R expression appeared to be increased on remaining dendrites and cell bodies and was dramatically increased on beaded dendrites (FIG. 2c) compared to controls. The widespread dendritic expression of PAF-R is consistent with a role for PAF in synaptic plasticity and excitotoxic injury. In addition to elevations in brain PAF concentration, increased PAF-R expression may further contribute to dendritic vulnerability in HAD.

cPAF Increases Vulnerability to Rapid Dendritic Beading following Elevated Synaptic Activity

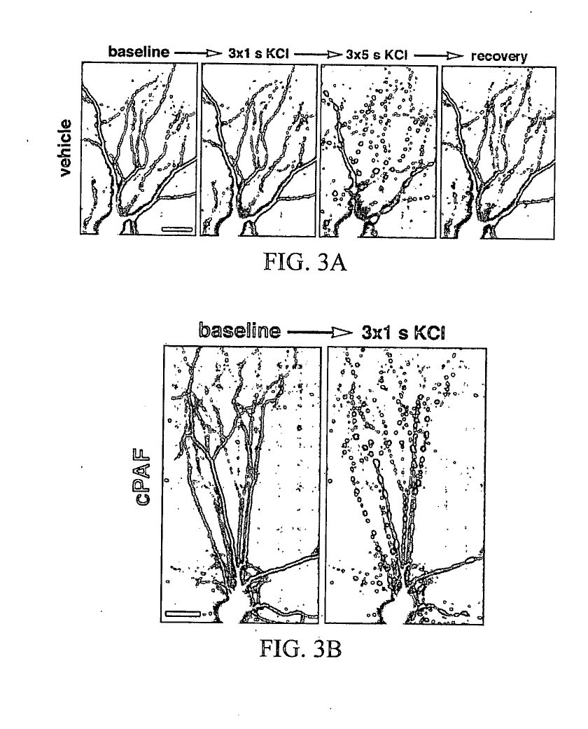

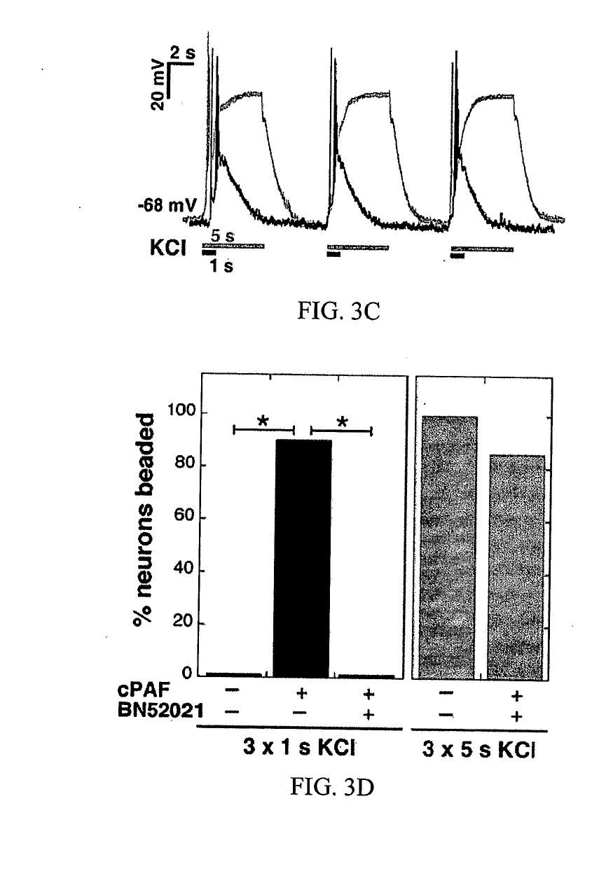

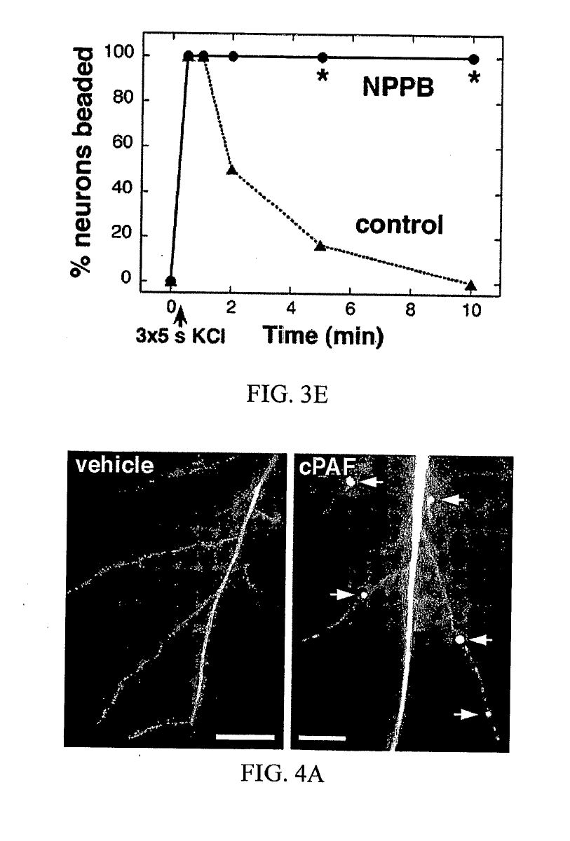

Because modulating spontaneous activity for 60 hours altered dendrite morphology in preliminary experiments and confounded cPAF effects, this was tested by measuring dendritic beading in cultured neurons after stimulating acute neurotransmitter release via depolarizing pulses of KCl. Control neurons showed no change in dendrite morphology following three 1 s pulses of KCl, a stimulus that causes NMDA receptor-dependent synaptic potentiation in dissociated cultures (39), but rapidly developed beading throughout their dendritic arbors (FIG. 3a) following 5 s pulses that evoked stronger and more prolonged depolarization (FIG. 3c). Acute (20 to 90 min) exposure to 130 nM cPAF lowered the threshold for dendritic beading, so that 90% of imaged cells beaded in response to three 1 s KCl pulses (FIGS. 3b, d; n=40 cells from 9 cultures, P<0.0001 vs. control). Beading was prevented by glutamate receptor antagonists CNQX (10 μM) and AP-5 (50 μM) (n=42 cells from 9 cultures, P<0.0001 vs. cPAF, 1 s KCl pulses), indicating that it resulted from KCl-induced glutamate release and not from depolarization alone.

Increased vulnerability of cPAF-exposed dendrites was blocked by pre-treatment with the PAF-R antagonist BN52021 (10 μM): no cells developed beading after 1 s KCl pulses (n=38 cells from 9 cultures, P<0.0001 vs. cPAF alone) and 86% beaded after 5 s pulses (FIG. 3d), similar to controls. Beading in both cPAF- and vehicle-exposed cultures developed rapidly (appearing within 10 s of the stimulus), began to recover within 1 to 2 minutes, and was fully resolved after 5 to 10 minutes in all imaged cells. Rapid recovery of KCl-induced beading was prevented by application of 5-nitro-2-(3-phenylpropylamino) benzoic acid (NPPB, 100 μM) (FIG. 3e; n=6 cells), a Cl⁻ channel blocker that inhibits volume-sensitive anion channels opened in response to neuronal swelling (40). This suggests that KCl-induced beading reflects acute dendritic swelling without lasting excitotoxicity.

### CPAF Promotes Dendritic Beading and Failure of Long-Term Potentiation in Hippocampal Slices

Whether cPAF disrupts synaptic plasticity by increasing vulnerability to activity-dependent dendritic injury in hippocampal slices was tested. dendritic beading and potentiation of excitatory post-synaptic potentials (EPSPs) in individual CAl pyramidal cells in acute rat hippocampal slices were measured, following high-frequency stimulation (BFS) of Schaffer collateral afferents. EPSPs were recorded by whole-cell patch clamp, and injected Alexa Fluor 568 hydrazide via the recording pipette for simultaneous dendrite imaging. In slice experiments, a higher (1 μM) dose of cPAF was used that was sufficient to augment excitatory transmission but was non-toxic to neurons in the slices: exposure to 1 μM cPAF for up to 7 hours did not affect baseline membrane potential (−63±3.6 mV cPAF, n=57 cells, vs.-63±3.4 mV vehicle, n=39 cells, P=0.99) or the ability to fire action potentials.

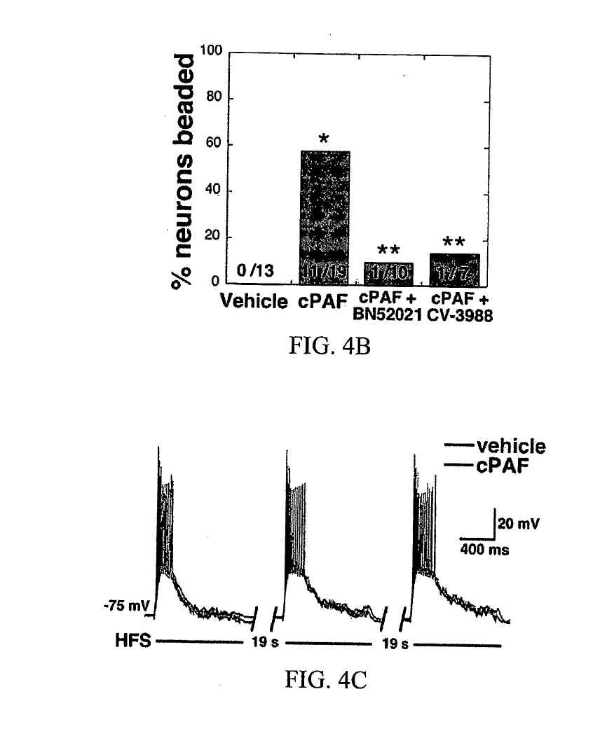

In control slices, HFS (three 1 s, 100 Hz trains, 20 s apart) elicited a robust, long-lasting potentiation of excitatory synaptic transmission (FIG. 4d): the rising slope of the postsynaptic potential increased 2.5-fold over baseline values following HFS (2.66±0.44 from 40 to 50 minutes. 71=13 slices, from 13 animals, P<0.001), and showed no decrement for the duration of the recording period (50 min). No vehicle-treated cells developed dendritic beading or other apparent change in dendrite morphology during the recording session (FIGS. 4a, b).

In contrast, exposure to 1 μM cPAF for 20 to 60 minutes prior to the recording session led to dendritic beading following HFS in 57% of recorded neurons (FIGS. 4a, b; 71=19 slices from 17 animals, P<0.001 vs. vehicle). HFS-induced dendritic beading was associated with a failure of synaptic potentiation (FIG. 4d): cPAF-exposed neurons that beaded showed no potentiation of EPSPs following HFS (0.84±0.12 relative to baseline from 40 to 50 min, n=11, P<0.01 vs. vehicle). On the other hand, cPAF-exposed cells that did not develop dendritic beading did undergo a long-lasting potentiation (1.60±0.26 relative to baseline from 40 to 50 min, n=8, P<0.05), although of a smaller magnitude than vehicle-treated cells. While exogenous PAF application has been proposed to occlude LTP in some experimental paradigms (28), this result indicates that 1 μM cPAF at most partially occluded LTP. Furthermore, EPSP potentiation was significantly different at all time points after HFS between cPAF-exposed cells that beaded and those that did not (P<0.05), strongly suggesting that failure of potentiation was a result of synaptic injury associated with dendritic beading.

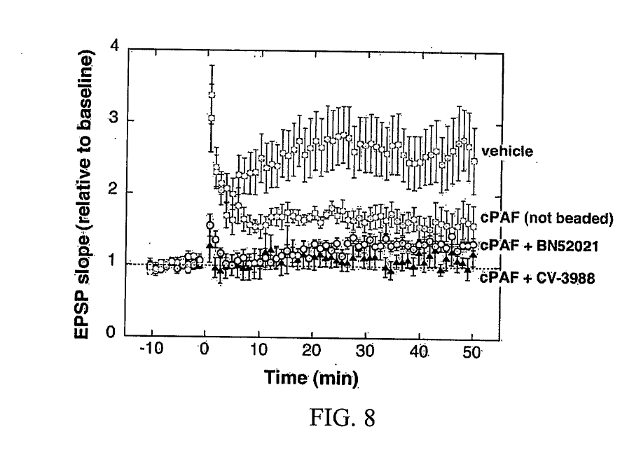

Dendritic beading in cPAF-exposed slices was largely prevented by structurally-distinct PAF-R antagonists (FIG. 4b): rates of beading following HFS in cPAF-exposed neurons were reduced to 14% by co-application of PAF analog CV-3988 (10 μM; n=7 slices from 3 animals, P<0.05 vs. cPAF alone), and to 10% by the structurally-unrelated antagonist BN52021 (2 μM; n=10 slices from 5 animals, P<0.05 vs. cPAF alone). PAF-R antagonists did not restore LTP in cPAF-exposed slices (FIG. 8), with a small potentiation of EPSPs (1.29±0.05 relative to baseline from 40 to 50 min, P<0.05) in BN52021-treated slices and no significant potentiation in CV-3988-treated slices (1.07±0.13 relative to baseline from 40 to 50 min, P=0.55).

cPAF-exposed neurons were depolarized for similar durations (693±296 ms cPAF, n=22, vs. 750±283 ms vehicle, n=15, P=0.56) and to similar extents (31.8:±11.9 mV cPAF vs. 27.8±5.6 mV vehicle, P=0.19) as controls during HFS (FIG. 4c), suggesting no gross differences in glutamate receptor activation between conditions.

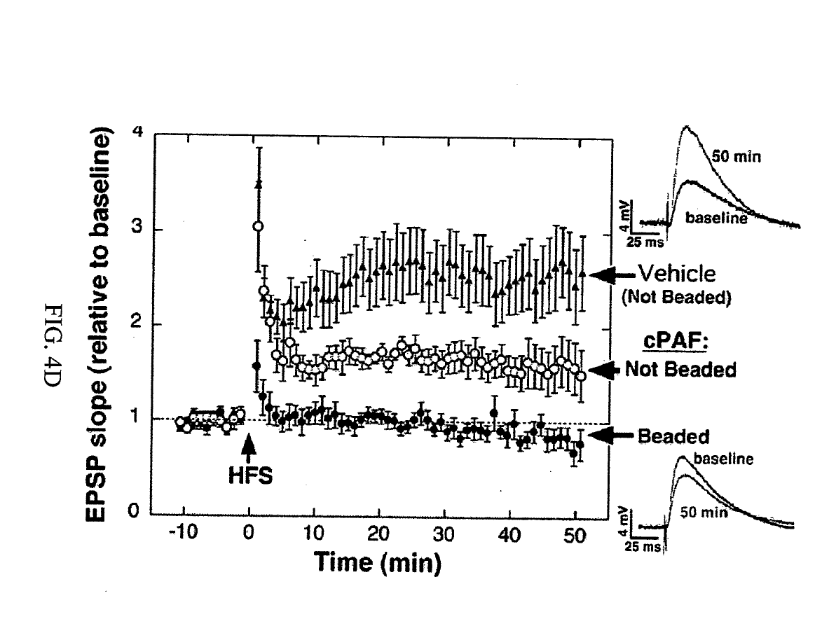

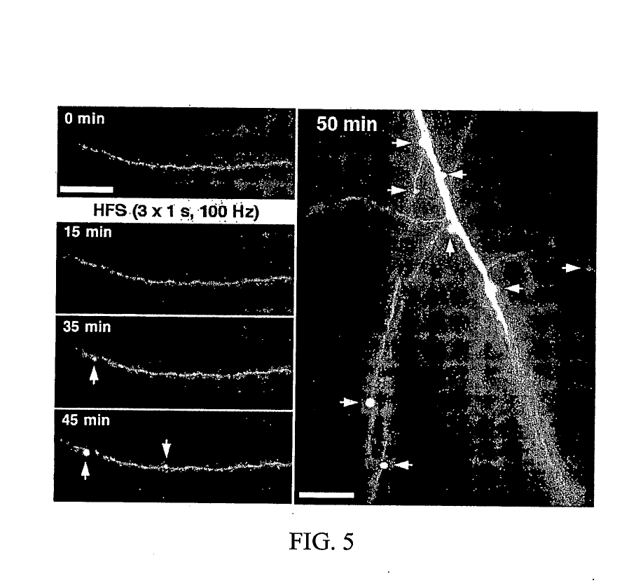

In cPAF-exposed cells, HFS-induced dendritic beading appeared after a delay, typically of 15-35 min, and often became more prominent throughout the recording session (FIG. 5). Beading developed at discrete locations and did not appear to disrupt dendritic spines in the intervening areas (FIGS. 4a, 5). Recovery of HFS-induced beading was never observed during the recording sessions (50 to 80 min post-HFS), though all neurons in the study remained viable throughout, maintaining negative membrane potentials and the ability to fire actions potential that overshot 0 mV. In addition, rapid dendritic swelling was not observed during or immediately after HFS in any dendrites, and no cells developed beading in the absence of HFS regardless of whether they were exposed to cPAF for up to 5 h or recorded in whole-cell mode for up to 90 min.

### Chemical Preconditioning Prevents Calcium- and Caspase-Dependent Dendritic Beading

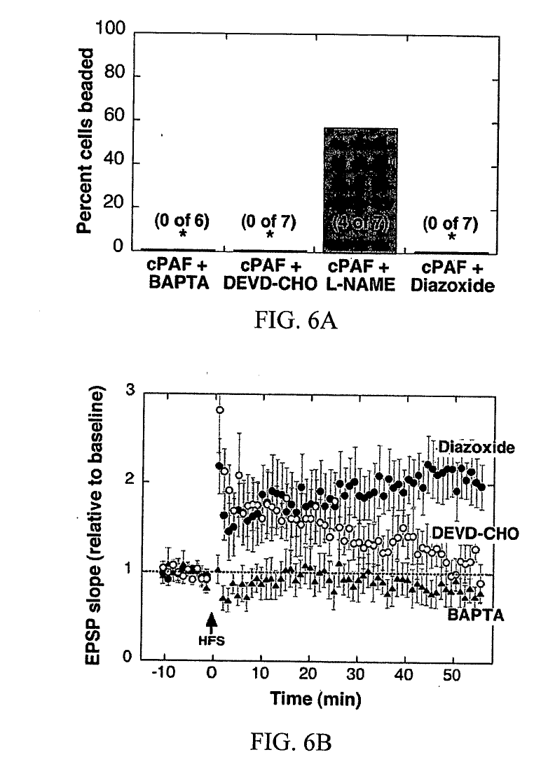

The delayed, progressive, and long-lasting appearance of beading following HFS led to the suspicion of calcium-mediated excitotoxic injury in the dendrites. This was tested by including 5 mM BAPTA in the recording pipette to chelate calcium in the post-synaptic cell of cPAF-exposed slices. BAPTA prevented dendritic beading (FIG. 6a; n=6 slices from 2 animals, P<0.05 vs. cPAF alone, FIG. 4b) and eliminated synaptic potentiation in all cells (FIG. 6b). Whether HFS-induced beading requires caspase activation in the post-synaptic cell was then tested by intracellular application of Ac-DEVD-CHO (10 μM), a caspase 3,6,7,8,10 inhibitor, via the recording pipette. Caspase inhibition prevented beading following HFS in all cPAF-exposed slices (FIG. 6a; n=7 slices from 4 animals, P<0.01 vs. cPAF alone, FIG. 4b), but failed to rescue LTP. After an initial 2-fold potentiation (FIG. 6b; 1.97±0.28 relative to baseline from 0 to 10 min, P<0.01) the EPSP slope gradually declined until it was not different from baseline at 40 to 50 min after HFS (1.22±0.19 relative to baseline, P>0.25). Because PAF has been reported to increase functional coupling between NMDA receptors and neuronal nitric oxide production (41), whether nitric oxide synthase inhibition by L-NAME (100 μM) prevents beading was tested. This offered no protection compared to treatment with cPAF alone (FIG. 4b), with 57% of L-NAME treated cells (FIG. 6a; n=7 slices from 3 animals) beading after HFS.

Finally, because HFS-induced dendritic beading appeared to be calcium- and caspase-mediated, whether diazoxide, an agonist of mitochondrial ATP-sensitive potassium channels, protects against beading was tested. Pretreatment with bath-applied diazoxide (30 μM) for 40 to 60 min prior to HFS in cPAF-exposed slices prevented dendritic beading (FIG. 6a; n=7 slices from 4 animals, P<0.01 vs. cPAF alone, FIG. 4b), and largely preserved LTP. Potentiation of EPSP slope appeared to be slightly blunted over the first 10 to 20 min following HFS, but strengthened until a 2-fold potentiation was maintained after 30 min (FIG. 6b; 2.10±0.27 relative to baseline from 40 to 50 min, P<0.01 vs. baseline and P<0.05 vs. all cPAF-exposed cells, FIG. 4d).

### Discussion

The data show that exposure to cPAF increases vulnerability to dendritic injury, including beading and spine loss that mimic the dendritic pathology of HAD. This can disrupt synaptic function by promoting calcium- and caspase-dependent dendritic beading and failure of EPSP enhancement following excitatory activity that normally induces LTP. In the presence of inflammatory mediators, physiologic synaptic activity can trigger dendritic injury and synaptic dysfunction. Thus there appears be an activity-dependent component to neuronal injury in HAD and perhaps other neurodegenerative diseases.

The time course and spatial distribution of dendritic beading in cPAF-exposed hippocampal slices (FIG. 5) suggests a different type of injury than that elicited by bath-applied KCl (FIG. 3), or by glutamate receptor agonists in previous studies (14, 15, 17). KCl and bath-applied agonists trigger beading that develops rapidly, affects nearly the entire dendritic arbor, and begins to recover soon after stimulus washout. Rapid, reversible beading has been shown to primarily reflect dendritic swelling, driven by large Na⁺ and Cl⁻ influxes and independent of calcium entry (14, 15). The present data show that NPPB, a Cl⁻ channel blocker that inhibits volume-sensitive currents crucial for reducing neuronal swelling (40), prevents rapid recovery of KCl-induced beading. In contrast, beading following high-frequency Schaffer collateral stimulation appears to reflect local excitotoxic injury in the dendrites: it is delayed, long-lasting, dependent on post-synaptic calcium and caspase activity, and disrupts discrete dendritic regions while leaving the majority of the arbor intact. Acute dendritic swelling was never seen following HFS; though bicuculline in the bath during EPSP recording experiments could have attenuated acute swelling by reducing Cl⁻ influx (14), similar results were seen in slices stimulated without bicuculline.

It is likely that activation of a small subset of synapses by Schaffer collateral stimulation, compared to widespread activation by bath-applied stimuli, limits ion influxes and thus avoids acute volume overload in the dendrites. This may have important functional implications. Rapid Na⁺, Cl⁻-dependent swelling has been proposed to protect neurons from high levels of extracellular glutamate during acute insults like trauma and ischemia: by transiently disrupting post-synaptic glutamate signaling, dendritic swelling may attenuate calcium-mediated excitotoxicity (17). In neuroinflammatory diseases such as HAD, on the other hand, the present data show that dendrites become vulnerable to localized excitotoxic damage in neighborhoods of elevated synaptic activity, which impairs function in a relatively synapse-specific manner.

Blockade of post-synaptic calcium signaling prevented dendritic beading following HFS, but did not improve synaptic function in cPAF-exposed slices (FIG. 6). Likewise, caspase activity inhibition prevented beading but failed to restore LTP.

In contrast, pre-treatment with diazoxide prevented dendritic beading while preserving LTP in cPAF-exposed slices (FIG. 6). These results show that chemical preconditioning is an effective strategy for improving synaptic function during chronic neuroinflammation.

Currently, use of antagonists such as memantine to inhibit excessive NMDA receptor activation (50) is the best-studied strategy to prevent excitotoxicity while preserving synaptic function in chronic neurodegenerative disease (51). Preconditioning represents an alternate or complementary strategy that can be especially valuable since the present results suggest that similar patterns of NMDA receptor activation can trigger either LTP or dendritic injury depending on the presence of inflammatory mediators.

### Methods

Golgi staining and immunohistochemistry. Paraformaldehyde-fixed post-mortem tissue obtained from HIV-1 seropositive patients who had undergone comprehensive neuropsychological testing as previously described (52) was studied. Tissue from mid-frontal cortex of patients with no neuropsychological impairment (n=5, post-mortem interval 8±3 h) and from patients with HAD (n=5, post-mortem interval 9±2 h) was compared. Tissue blocks were trimmed to 2 mm³, silver-impregnated with the rapid Golgi method and sectioned at 100 μm as previously described (7). For immunohistochemical analysis, 40 g/m vibratome sections were incubated overnight with antibodies against PAF-R (Cayman Chemical, 1:250), detecting them with horseradish peroxidase and the Tyramide Signal Amplification-Direct (Red) system (Perkin Ehner Life and Analytical Sciences) or diaminobenzidine, followed in some studies by antibodies against MAP2 (Chemicon, 1:100) detected with FITC-conjugated horse anti-mouse IgG (1:75) (Vector Laboratories Inc.). To control for non-specific binding PAF-R antibody was incubated overnight with a PAF-R-derived blocking peptide (Cayman, 1:20) prior to incubation with tissue sections. Slide-mounted sections were analyzed with laser scanning confocal microscopy (MRC1024, BioRad Laboratories). All sections were processed simultaneously and experiments were repeated to assess reproducibility. Studies in patients were conducted according to the Helsinki declaration and with approval from the University of California San Diego Human Subjects Review Board. All patients provided informed consent prior to inclusion in the study and were identified by number, not name.

Primary hippocampal cultures and dendrite imaging. Dissociated hippocampal cultures from embryonic (E18) rats were prepared as previously described (53), plated on coverslips coated with poly-D-lysine and mouse laminin (reagents from Sigma-Aldrich unless otherwise noted) in Neurobasal plus B-27 media (GIBCO). After one week, 53 mM NaCl was added (to match solutions for physiological experiments) and antioxidants were removed from the media. Experiments were repeated using cultures from multiple dissections. Rodents were housed and treated in compliance with University of Rochester Committee on Animal Resources and NIH policies, and New York State and federal statutes.

At 20-24 days in vitro (DIV) neurons were transfected with EYFP (Clontech) or mRFP-1 (kind gift of Roger Tsien) vectors driven by CMV promoter, in a 1:2 ratio with Lipofectamine 2000 (Invitrogen). Serial fluorescence images of individual, live neurons (23-30 DIV) before and after exposure to 130 nM cPAF (Biomol) and/or synaptic stimulation by KCl were captured. For 60-hour exposure experiments, a 10 mM stock solution of cPAF (in EtOH) or vehicle was diluted into culture media. Neuronal survival was measured by Hoechst nuclear staining and propidium iodide exclusion.

For acute KCl stimulation experiments, coverslips were perfused in a custom-made chamber (200 μl) with bath solution (in mM: NaCl 139.5, KCl 2.5, CaCl₂ 2, MgCl₂ 1, glucose 24, HEPES 5, glycine 0.01, pH 7.3) at 1 mmin, and exposed cultures to vehicle or cPAF for 20-90 min prior to stimulation. KCl (90 mM) was locally applied over the entire dendritic arbor of EYFP- or mRFP-expressing neurons in three 1- or 5-s pulses (10 s apart) using an 8-channel drug delivery system (ALA Scientific Instruments). In some experiments BN52021 (Biomol) or CNQX and AP-5 were added.

Imaging and LTP in acute hippocampal slices. Brains were removed from 17-30 day-old male Sprague-Dawley rats anesthetized with ketamine (180 μg/g), submerged them in ice-cold solution (in mM: NaCl 125, KCl 5, NaH₂PO₄ 1.25, NaHCO₃ 28, CaCl₂ 0.5, MgCl₂ 4, D-glucose 25, kynurenic acid 1, bubbled with 95% O₂/5% CO₂), and cut 250 μm coronal slices using a vibroslice. Slices recovered in artificial cerebrospinal fluid (aCSF, in mM: NaCl 125, KCl 2.5, NaH₂PO₄ 1.25, NaHCO₃ 25, CaCl₂ 2, MgCl₂ 1, D-glucose 25, bubbled with 95% O₂/5% CO₂) for >90 min prior to experiments.

Slices were transferred to a custom-made recording chamber perfused (1 ml/min) with aCSF containing 1 μM cPAF or vehicle. Membrane potentials from CA1 pyramidal neurons were recorded via whole-cell patch clamp, using 4-6 MΩ electrodes (filled with, mM: KCl 20, potassium gluconate 130, EGTA 0.5, HEPES10, MgSO₄ 2, ATP 2.5, GTP 0.5, pH 7.3) and a Multiclamp 700A amplifier with pClamp 9 software (Axon Instruments). Afferents were stimulated by constant current pulses (200 μs) via a bipolar stimulating electrode 50-200 μm away in the stratum radiatum. 10 μM bicuculline were included in the aCSF to isolate EPSPs, and adjusted stimulating intensity to elicit EPSPs 30% of maximal. After >10 min of test pulses (every 15 s), 90=gave a high-frequency stimulus (HFS; three 1 s, 100 Hz trains, 20 s apart, at test-pulse intensity), then resumed test pulses for ≧50 min. In some experiments L-NAME, diazoxide (Alexis Biochemical Corp.), BN52021, or CV-3988 (Biomol) were included in the aCSF throughout the recording, or BAPTA or Ac-DEVD-CHO in the electrode solution. Rising EPSP slope was used instead of peak amplitude as an index of synaptic efficacy to, avoid complication by action potentials evoked by some EPSPs after HFS. Amplitude (maximal sustained depolarization) and duration (at >25% maximal amplitude) of depolarization during HFS were also quantified.

Fluorescent images of dendrites from recorded cells were collected before and after HFS, after allowing 30 μM Alexa Fluor 568 hydrazide (Molecular Probes) in the recording electrode to fill the cell for >20 min. Using an external shutter to limit light exposure, image frames were captured at multiple focal planes and combined optimally-focused portions of these frames to display a composite image.

Dendrite morphology. For in vitro experiments pre- and post-exposure images of the same cells were compared. A neuron was considered beaded if its dendrites developed any focal swellings during the experiments (10, 14), though multiple dendrites were typically involved. Changes in spine number were determined by counting spines on the same dendrites before and after treatment.

Statistics. Differences in frequencies of dendritic beading were analyzed by Chi square, and changes in dendritic spine number by paired t-test. EPSP slope data were pooled over 10-minute intervals and used two-tailed t-tests to compare between groups or time points. A 0.05 significance level was used for all tests.

### Example 2

### Activity-Dependent Mitochondrial Stress Determines Synaptic Fate

Specific patterns of neural activity can trigger elaborate cellular programs to adjust both synaptic morphology and the strength of synaptic transmission. The present experiments show that when levels of the inflammatory mediator platelet-activating factor (PAF) are elevated in the brain, identical synaptic activity can instead activate an alternate program leading to local dendritic injury and disruption of synaptic function. This likely changes the rules of synaptic learning that govern information processing and the formation of neural circuitry, and provides insight into how chronic inflammatory diseases can disrupt neurologic function even at early disease stages, before irreversible neuronal dysfunction and loss.

Protecting synaptic function at these stages can preserve neurologic function. The present data highlight that synaptic protection is not straightforward: both long-term potentiation (LTP) and excitotoxic dendritic beading can be triggered by similar synaptic activity, and appear to involve much of the same cellular machinery. Thus, many treatments that protected against dendritic beading also impaired LTP in hippocampal slices exposed to cPAF. In contrast, pre-treatment with diazoxide, a mitochondrial K_{ATP} channel agonist that induces preconditioning, prevented beading and preserved LTP. In the presence of inflammatory mediators, chemical preconditioning appears to re-direct the synaptic response from excitotoxicity back towards plasticity without blocking cell signaling mechanisms that are essential for both processes. Thus, chemical preconditioning is a promising approach to preserving synaptic structure and function in HAD and other neurodegenerative diseases with a chronic inflammatory component.

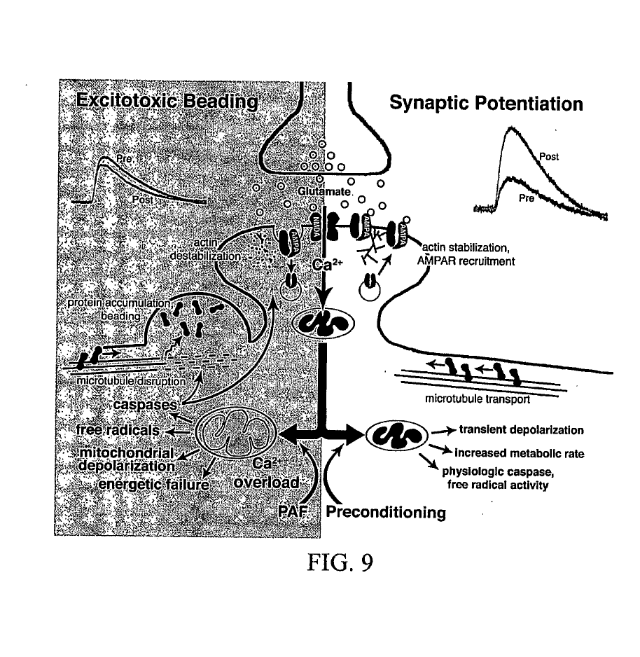

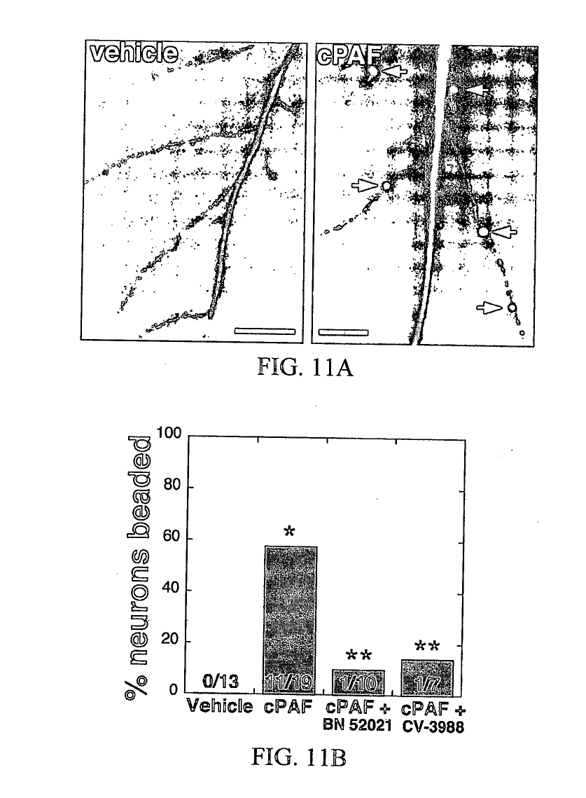

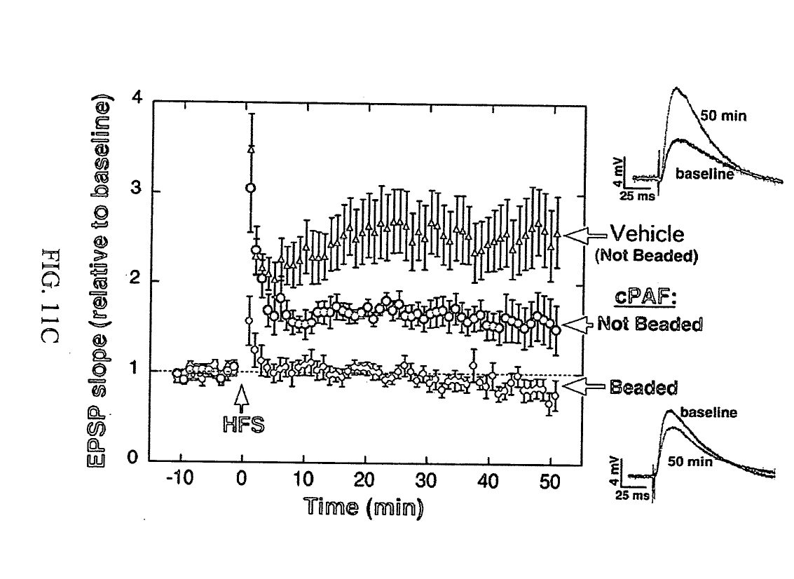

The degree of mitochondrial stress following excitatory stimulation dictates whether synapses proceed towards LTP or excitotoxic injury. The present data indicate that this decision is made quickly: excitatory post-synaptic potentials (EPSPs) recorded from neurons that went on to develop dendritic beading returned to baseline magnitude within minutes of high-frequency stimulation (HFS), diverging sharply from those of uninjured neurons that achieved a significant early potentiation (FIG. 11). Although activity-dependent dendritic beading and the maintained phase of LTP take 30 minutes or more to fully develop, synapses appear to be directed toward one fate or the other much sooner after stimulation. Thus, post-synaptic mitochondria, poised to respond to calcium influx immediately following HFS, play a critical role in deciding whether to activate cellular programs for physiologic plasticity or those for excitotoxicity (FIG. 9).

Post-synaptic calcium influx has long been recognized as necessary for both LTP and excitotoxicity (59, 60). After entering the dendrites and spines, calcium in the cytosol activates a myriad of enzymes and signaling cascades that are critical for LTP, including calnodulin-dependent kinase II (CaMKII) that strengthens synaptic transmission via phosphorylation and recruitment of AMPA receptors (61, 62, 63). Incoming calcium can also be taken up by nearby mitochondria, driven by the electrochemical gradient across the inner mitochondrial membrane. Uptake of small amounts of calcium can cause a mild, transient mitochondrial depolarization and may trigger a physiologic increase in rates of oxidative metabolism (64, 65, 66). Excessive calcium uptake, in contrast, can overload the mitochondria and cause swelling, marked depolarization, metabolic failure, toxic free radical production and caspase activation (67). Experiments with cultured neurons suggest that excessive mitochondrial calcium uptake, and not the concentration of cytosolic calcium, triggers excitotoxic injury: when mitochondrial uptake is blocked, neurons can survive normally-lethal glutamate exposures and very high cytosolic calcium concentrations with little lasting effect (68, 69).

Thus, in hippocampal slices following HFS, a rise in post-synaptic cytosolic calcium likely activates CaMKII and other calcium-dependent enzymes to initiate synaptic potentiation. Under normal conditions, post-synaptic mitochondria take up a small amount of calcium, triggering an increase in metabolic rate and a mild depolarization that contribute to the further development of LTP (FIG. 9). Increased ATP production at activated synapses likely powers energy-intensive processes such as local protein synthesis, transport of synaptic proteins and organelles along microtubules, and post-synaptic actin polymerization that provide the building blocks for stronger synaptic transmission (70, 71). Accelerated oxidation and mild depolarization also likely provide the physiologic free radical production and caspase activation that have been shown to be required for maintenance of LTP (72, 73) cPAF promotes excitotoxic dendritic beading by increasing mitochondrial calcium uptake following HFS. cPAF and other pro-inflammatory molecules have been shown to hyperpolarize mitochondria at baseline (74), increasing the electrochemical gradient for calcium uptake. This likely predisposes post-synaptic mitochondria to calcium overload following HFS, with subsequent damage that disrupts LTP and leads to dendritic beading. Mitochondrial swelling and release of pro-apoptotic molecules such as cytochrome c can trigger activation of caspases, which mediate the early failure of synaptic strengthening likely by cleaving glutamate receptors and post-synaptic actin (75, 76). Caspases can also cleave nearby microtubules, leading to development of long-lasting dendritic beading by undermining the dendrite's structural integrity and by derailing the transport of proteins and organelles, which accumulate locally. Severe mitochondrial depolarization with local energy depletion and free radical production likely add to the structural injury and disrupted protein trafficking. This likely contributes to the failure of late-phase LTP in beaded neurons, and may ultimately lead to a net loss of AMPA receptors from the synapse and weakened neurotransmission.

The present experiments measure changes in mitochondrial membrane potential during and after HFS in hippocampal slices using the rhodamine 123, a cationic dye that distributes across the mitochondrial membrane in a voltage-sensitive manner. When loaded into hippocampal slices at a dose of 26 μM, rhodamine 123 accumulates in the mitochondria at high concentrations that causes its fluorescence to quench; if the mitochondria are subsequently depolarized, rhodamine 123 is released into the cytoplasm and its fluorescent signal increases. Mitochondrial hyperpolarization, conversely, drives more dye from the cytoplasm into the mitochondria, increasing quenching and reducing the signal. Reliability of this interpretation was confirmed by control experiments using FCCP and oligomycin to respectively depolarize and hyperpolarize the mitochondria. Compared to other fluorescent indicators of mitochondrial potential such as TMRM and JC-1, rhodamine 123 provided the best penetration into the full thickness of the hippocampal slices and the most stable signal with repeated imaging under baseline, unstimulated conditions.

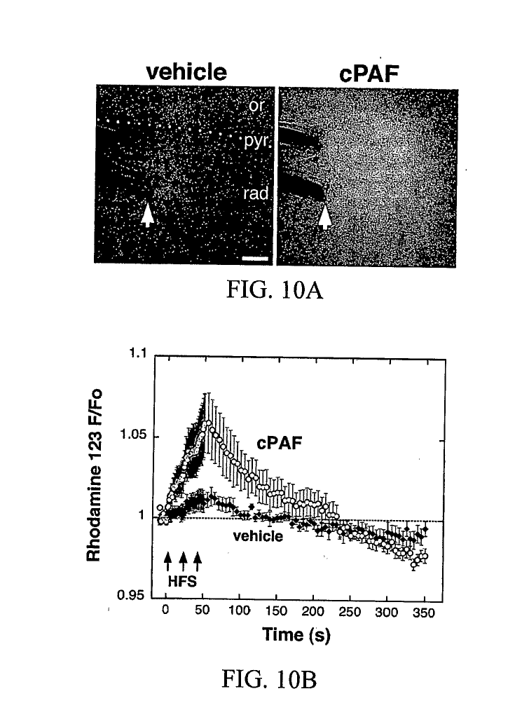

The data have demonstrated a very small, transient increase in rhodamine 123 fluorescence following HFS in control slices, reaching a peak of 1.37±0.46% greater than baseline 45 s after the start of HFS (FIG. 10; n=7 slices from 4 animals). In slices exposed to 1 μM cPAF the increase was four-fold larger, peaking at 5.90±1.86% above baseline 55 s after the start of HFS (FIG. 10; n=8 slices from 4 animals). The greater activity-dependent mitochondrial depolarization suggests that although cPAF does not appear to affect the calcium influx into the post-synaptic cytosol, it may augment calcium uptake by the mitochondria. Because this technique measures rhodamine 123 from all cell types in the slice, changes in fluorescence reflect the state of pre-synaptic and glial mitochondria in addition to post-synaptic mitochondria. Thus even a severe depolarization of a subset of post-synaptic mitochondria following HFS may only cause a small change in the overall fluorescent signal. Further experiments using NMDA receptor antagonists to block post-synaptic calcium influx, or metabotropic glutamate receptor antagonists to block activity-dependent rises in glial calcium (77) can determine whether the observed change in rhodamine 123 signal is predominantly due to depolarization of dendritic mitochondria.

Nevertheless, the four-fold greater increase in rhodamine 123 fluorescence following HFS in cPAF-treated compared to control slices supports the view that activity-dependent excitotoxic beading is associated with increased mitochondrial stress. This can be further investigated using indicators sensitive to mitochondrial calcium concentration and free radical activity. Mitochondrial stress dictates whether activated synapses undergo LTP or excitotoxic injury. Thus, the present methods of assessing mitochondrial function following HFS presents a model system for identifying additional specific agents that can provide effective synaptic protection in neurodegenerative disease with an inflammatory component.

### Example 3

### Use of Mouse Model to Demonstrate in Vivo Efficacy of K⁺ ATP Channel Agonists

A SCID mouse model of HIV is used to study HIV progression, and HAD in particular. The methods disclosed herein are tested using this model.

### Primary Isolation and HIV-1 Infection of Monocyte-Derived Macrophages (MDM).

Monocytes are obtained from leukopaks of HIV-1, 2 and hepatitis B seronegative donors and purified by countercurrent centrifugal elutriation. Cells are cultured with 1000 U/ml of highly purified recombinant human macrophage colony stimulating factor (MCSF) (from Genetics Institute, Inc., Cambridge, Mass.), and MDM are infected with HIV-1_{ADA} (a macrophage tropic viral strain) at multiplicity of infection (MOI) of 0.01.

### Severe Combined Immunodeficient Disease (SCID) Mouse Model of HIVE.

Four week old male C.B-17 SCID mice, purchased from the Jackson Laboratory (Bar Harbor, Me., USA), are maintained in sterile microisolator cages. HIV-1_{ADA}-infected MDM (3×10⁵ cells in 5 ml) are injected intracranially (i.c.) on the seventh day following viral infection. One day prior to MDM placement, diazoxide, nicorandil or other K ATP channel openers such as NN414 (78) are administered either intraperitoneally or intrathecally and then animals are sacrificed at day 8 (peak of inflammation and neuronal injury). Control animals receive the vehicle formulation for diazoxide, nicorandil or other K ATP channel openers. Animals are sacrificed 7 days after MDM placement.

Analyses of Brain Tissue from Treated Animal.

Histopathology and image analysis. Brain tissue is collected at necropsy, fixed in 4% phosphate-buffered paraformaldehyde and embedded in paraffin or frozen for later use. Blocks are cut to identify the injection site. For each mouse, 30-100 serial (5-mm-thick) sections are cut from the injection site and at the level of the hippocampus. Immunohistochemical staining followed a basic indirect protocol. Alternatively, brains are frozen after fixation and 30 mm sections are prepared for immunofluorescent staining. Antibodies to vimentin intermediate filaments (clone 3B4, Dako) are used for detection of human cells in mouse brain. Murine microglia are identified by rabbit polyclonal antibodies to ionized calcium-binding adapter molecule 1 (lba-1, 1:500, Wako, Richmond, Va.) and Griffonia simplicifolia Lectin I-Isolectin B4. Astrocytes are labeled to antibodies for glial fibrillary acidic protein (GFAP, 1:2000, Dako). Neuron-specific nuclear protein (NeuN), microtubule-associated protein 2 (MAP-2) and synaptophysin (SYP) are used for neuronal detection. Antibodies to HIV-1 p24 antigen (Dako) are applied to determine the number of infected cells. Immature neurons are localized by antibodies to polysialated neuronal cell adhesion molecule (PSA-NCAM, mouse IgM, 1:1000, provided by Dr. T. Seki, Jutendo University School of Medicine, Tokyo, Japan). All paraffin-embedded sections are counterstained with Mayer's hematoxylin. Deletion of primary antibody served as a control. Tissue examination is performed with a Nikon Eclipse E800 (Nikon Instruments Inc., Melville, N.Y.). Images are obtained by Optronics digital camera (Buffalo Grove, Ill.) with MagnaFire 2.0 software (Goleta, Calif.) and processed by Adobe Photoshop 7.0 software.

Assays of Neuronal Apoptosis. TUNEL assay is performed using the In Situ Cell Death Detection Kit, TMR Red (Roche Diagnostic Corporation, Indianapolis, Ind.) following the manufacturer's protocol. Apoptotic cells are identified with TUNEL, which detects the DNA fragmentation that is a characteristic feature of apoptotic cells. The cells are viewed under a fluorescence microscope and the total number of bright green nuclei in the field is counted. Using the same field of view, MAP-2 positive staining length (pixel) is used to normalize apoptosis by TUNEL/MAP-2. Neuronal apoptosis index is calculated by dividing the number of counted TUNEL positive green nuclei by length (pixel) of MAP-2.

Western blot assays. Two-millimeter sections that included the site of injection are used for extraction of proteins. Tissue sections corresponding to the site of injection in the contralateral hemisphere serve as controls. The brain is homogenized in lysis buffer containing 50 mM Tris-HCl, pH 7.4, 150 mM NaCl, 2 mM EDTA, 1% NP40, aprotinin, bestatin, leupeptin, pepstatin A, aminoethyl benzenesulfonylfluoride, and E-64. Proteins are electrophoretically separated on SDS-polyacrylamide gels and transferred onto polyvinyldifluoridene membranes. Membranes are incubated with primary antibodies to Tau 5, Phospho-Tau Ser²⁰², □-catenin, Phospho-□-catenin Ser33, 37, GSK-3□, MAP-2 or actin and □-tubulin. Horseradish peroxidase conjugated secondary antibodies are used and membranes are treated with chemiluminiscent substrate, and then exposed to X-ray film. Images are digitized with a Molecular Dynamics densitometer (Molecular Dynamics, Inc.) and protein levels are expressed as a ratio to actin or tubulin. Data are analyzed using Excel with Student t-test for comparisons. All statistics are presented as mean±SEM.

Electrophysiological tests. Seven days after injection, brains are quickly removed from the cranial cavities. Hippocampi ipsilateral to the injection site are separated and placed in ice-cold (4° C.) oxygenated artificial cerebral spinal fluid (ACSF) prior to sectioning. The ability of high frequency stimulation (HFS) to induce LTP in the CAl region of the hippocampus is examined after 30-min. LTP is induced by weak tetanic stimulation (10 events at 100 Hz) observed for 60 min as previously described (Anderson, et al. 2003). Results from slices with large fluctuation (>2 standard deviations, S.D.) are rejected.

The SCID mouse model is an accepted model for neuroaids (79, 80, 81).

### REFERENCES

1. Masliah, E., Heaton, R. K., Marcotte, T. D., Ellis, R. J., Wiley, C. A., Mallory, M., Achim, C. L., McCutchan, J. A., Nelson, J. A., Atkinson, J. H., et al. 1997. Dendritic injury is a pathological substrate for human immunodeficiency virus-related cognitive disorders. HNRC Group. The HIV Neurobehavioral Research Center. Ann Neurol 42:963-972.
2. Everall, I. P., Glass, J. D., McArthur, J., Spargo, E., and Lantos, P. 1994. Neuronal density in the superior frontal and temporal gyri does not correlate with the degree of human immunodeficiency virus-associated dementia. Acta Neuropathol (Berl) 88:538-544.
3. Adle-Biassette, H., Chretien, F., Wingertsmann, L., Hery, C., Ereau, T., Scaravilli, F., Tardieu, M., and Gray, F. 1999. Neuronal apoptosis does not correlate with dementia in HIV infection but is related to microglial activation and axonal damage. Neuropathol Appl Neurobiol 25:123-133.
4. DeKosky, S. T., and Scheff, S. W. 1990. Synapse loss in frontal cortex biopsies in Alzheimer's disease: correlation with cognitive severity. Ann Neurol 27:457-464.
5. Terry, R. D., Masliah, E., Salmon, D. P., Butters, N., DeTeresa, R., Hill, R., Hansen, L. A., and Katzman, R. 1991. Physical basis of cognitive alterations in Alzheimer's disease: synapse loss is the major correlate of cognitive impairment. Ann Neurol 30:572-580.
6. Masliah, E., Ge, N., Achim, C. L., Hansen, L. A., and Wiley, C. A. 1992. Selective neuronal vulnerability in HIV encephalitis. J Neuropathol Exp Neurol 51:585-593.
7. Masliah, E., Ge, N., Morey, M., DeTeresa, R., Terry, R. D., and Wiley, C. A. 1992. Cortical dendritic pathology in human immunodeficiency virus encephalitis. Lab Invest 66:285-291.
8. Ferrer, I., Guionnet, N., Cruz-Sanchez, F., and Tunon, T. 1990. Neuronal alterations in patients with dementia: a Golgi study on biopsy samples. Neurosci Lett 114:11-16.
9. Masliah, E., Mallory, M., Hansen, L., DeTeresa, R., Alford, M., and Terry, R. 1994. Synaptic and neuritic alterations during the progression of Alzheimer's disease. Neurosci Lett 174:67-72.
10. Park, J. S., Bateman, M. C., and Goldberg, M. P. 1996. Rapid alterations in dendrite morphology during sublethal hypoxia or glutamate receptor activation. Neurobiol Dis 3:215-227.
11. Ivins, K. J., Bui, E. T., and Cotman, C. W. 1998. Beta-amyloid induces local neurite degeneration in cultured hippocampal neurons: evidence for neuritic apoptosis. Neurobiol Dis 5:365-378.
12. Mattson, M. P., Keller, J. N., and Begley, J. G. 1998. Evidence for synaptic apoptosis. Exp Neurol 153:35-48.
13. White, A. R., Guirguis, R., Brazier, M. W., Jobling, M. F., Hill, A. F., Beyreuther, K., Barrow, C. J., Masters, C. L., Collins, S. J., and Cappai, R. 2001. Sublethal concentrations of prion peptide PrP106-126 or the amyloid beta peptide of Alzheimer's disease activates expression of proapoptotic markers in primary cortical neurons. Neurobiol Dis 8:299-316.
14. Hasbani, M. J., Hyrc, K. L., Faddis, B. T., Romano, C., and Goldberg, M. P. 1998. Distinct roles for sodium, chloride, and calcium in excitotoxic dendritic injury and recovery. Exp Neurol 154:241-258.
15. Al-Noori, S., and Swami, J. W. 2000. A role for sodium and chloride in kainic acid-induced beading of inhibitory interneuron dendrites. Neuroscience 101:337-348.
16. Hasbani, M.J., Schlief, M. L., Fisher, D. A., and Goldberg, M. P. 2001. Dendritic spines lost during glutamate receptor activation reemerge at original sites of synaptic contact. J Neurosci 21:2393-2403.
17. Ikegaya, Y., Kim, J. A., Baba, M., Iwatsubo, T., Nishiyama, N., and Matsuki, N. 2001. Rapid and reversible changes in dendrite morphology and synaptic efficacy following NMDA receptor activation: implication for a cellular defense against excitotoxicity. J Cell Sci 114:4083-4093.
18. Brendza, R. P., Bacskai, B. J., Cirrito, J. R., Simmons, K. A., Skoch, J. M., Klunk, W. E., Mathis, C. A., Bales, K. R., Paul, S. M., Hyman, B. T., et al. 2005. Anti-Abeta antibody treatment promotes the rapid recovery of amyloid-associated neuritic dystrophy in PDAPP transgenic mice. J Clin Invest 115:428-433.
19. Zhu, B., Luo, L., Moore, G. R., Paty, D. W., and Cynader, M. S. 2003. Dendritic and synaptic pathology in experimental autoimmune encephalomyelitis. Am J Pathol 162:1639-1650.
20. Puel, J. L., Ruel, J., Gervais d'Aldin, C., and Pujol, R. 1998. Excitotoxicity and repair of cochlear synapses after noise-trauma induced hearing loss. Neuroreport 9:2109-2114.
21. Gendeiman, H. E., Zheng, J., Coulter, C.L., Ghorpade, A., Che, M., Thylin, M., Rubocki, R., Persidsky, Y., Hahn, F., Reinhard, J., Jr., et al. 1998. Suppression of inflammatory neurotoxins by highly active antiretroviral therapy in human immunodeficiency virus-associated dementia. J Infect Dis 178:1000-1007.
22. Thuniher, M. M., Schindler, E. G., Thurnher, S. A., Pemerstorfer-Schon, H., Kleibl-Popov, C., and Rieger, A. 2000. Highly active antiretroviral therapy for patients with AIDS dementia complex: effect on MR imaging findings and clinical course. AJNR Am J Neuroradiol 21:670-678.
23. Genis, P., Jett, M., Beruton, E. W., Boyle, T., Gelbard, H. A., Dzenko, K., Keane, R. W., Resnick, L., Mizrachi, Y., and Volsky, D. J. 1992. Cytokines and arachidonic metabolites produced during human immunodeficiency virus (HIV)-infected macrophage-astroglia interactions: implications for the neuropathogenesis of HIV disease. J Exp Med 176:1703-1718.
24. Perry, S. W., Hamilton, J. A., Tjoelker, L. W., Dbaibo, G., Dzenko, K. A., Epstein, L. G., Hannun, Y., Whittaker, J. S., Dewhurst, S., and Gelbard, H. A. 1998. Platelet-activating factor receptor activation. An initiator step in HIV-1 neuropathogenesis. J Biol Chem 273:17660-17664.
25. Giulian, D., Vaca, K., and Noonan, C. A. 1990. Secretion of neurotoxins by mononuclear phagocytes infected with HIV-1. Science 250:1593-1596.
26. Aihara, M., Ishii, S., Kume, K., and Shimizu, T. 2000. Interaction between neurone and microglia mediated by platelet-activating factor. Genes Cells 5:397-406.
27. Clark, G. D., Happel, L. T., Zorumski, C.F., and Bazan, N. G. 1992. Enhancement of hippocampal excitatory synaptic transmission by platelet-activating factor. Neuron 9:1211-1216.
28. Wieraszko, A., Li, G., Komecki, E., Hogan, M. V., and Ehrlich, Y. H. 1993. Long-term potentiation in the hippocampus induced by platelet-activating factor. Neuron 10:553-557.
29. Kato, K., Clark, G. D., Bazan, N. G., and Zorumski, C.F. 1994. Platelet-activating factor as a potential retrograde messenger in CA1 hippocampal long-term potentiation. Nature 367:175-179.
30. Izquierdo, I., Fin, C., Schmitz, P. K., Da Silva, R. C., Jerusalinsky, D., Quillfeldt, J. A., Ferreira, M. B., Medina, J. H., and Bazan, N. G. 1995. Memory enhancement by intrahippocampal, intraamygdala, or intraentorhinal infusion of platelet-activating factor measured in an inhibitory avoidance task. Proc Natl Acad Sci USA 92:5047-5051.
31. Teather, L. A., Packard, M. G., and Bazan, N. G. 1998. Effects of posttraining intrahippocampal injections of platelet-activating factor and PAF antagonists on memory. Neurobiol Learn Mem 70:349-363.
32. Gelbard, H. A., Nottet, H. S., Swindells, S., Jett, M., Dzenko, K. A., Genis, P., White, R., Wang, L., Choi, Y. B., and Zhang, D. 1994. Platelet-activating factor: a candidate human immunodeficiency virus type 1-induced neurotoxin. J Virol 68:4628-4635.
33. Lindsberg, P. J., Yue, T. L., Frerichs, K. U., Hallenbeck, J. M., and Feuerstein, G. 1990. Evidence for platelet-activating factor as a novel mediator in experimental stroke in rabbits. Stroke 21:1452-1457.
34. Kumar, R., Harvey, S. A., Kester, M., Hanahan, D. J., and Olson, M. S. 1988. Production and effects of platelet-activating factor in the rat brain. Biochim Biophys Acta 963:375-383.
35. Ogden, F., DeCoster, M. A., and Bazan, N. G. 1998. Recombinant plasma-type platelet-activating factor acetylhydrolase attenuates NMDA-induced hippocampal neuronal apoptosis. J Neurosci Res 53:677-684.
36. Xu, Y., and Tao, Y. X. 2004. Involvement of the NMDA receptor/nitric oxide signal pathway in platelet-activating factor-induced neurotoxicity. Neuroreport 15:263-266.
37. Ho, Y. S., Swenson, L., Derewenda, U., Serre, L., Wei, Y., Dauter, Z., Hattori, M., Adachi, T., Aoki, J., Arai, H., et al. 1997. Brain acetylhydrolase that inactivates platelet-activating factor is a G-protein-like trimer. Nature 385:89-93.
38. Mori, M., Aihara, M., Kume, K., Hamanoue, M., Kohsaka, S., and Shimizu, T. 1996. Predominant expression of platelet-activating factor receptor in the rat brain microglia. J Neurosci 16:3590-3600.
39. Fitzjohn, S. M., Pickard, L., Duckworth, J. K., Molnar, E., Henley, J. M., Collingridge, G. L., and Noel, J. 2001. An electrophysiological characterisation of long-term potentiation in cultured dissociated hippocampal neurones. Neuropharmacology 41:693-699.
40. Patel, A. J., Lauritzen, I., Lazdunski, M., and Honore, E. 1998. Disruption of mitochondrial respiration inhibits volume-regulated anion channels and provokes neuronal cell swelling. J Neurosci 18:3117-3123.
41. Xu, Y., Zhang, B., Hua, Z., Johns, R. A., Bredt, D. S., and Tao, Y. X. 2004. Targeted disruption of PSD-93 gene reduces platelet-activating factor-induced neurotoxicity in cultured cortical neurons. Exp Neurol 189:16-24.
42. K is, B., Rajapakse, N. C., Snipes, J. A., Nagy, K., Horiguchi, T., and Busija, D. W. 2003. Diazoxide induces delayed pre-conditioning in cultured rat cortical neurons. J Neurochem 87:969-980.
43. Nagy, K., K is, B., Rajapakse, N. C., Bari, F., and Busija, D. W. 2004. Diazoxide preconditioning protects against neuronal cell death by attenuation of oxidative stress upon glutamate stimulation. J Neurosci Res 76:697-704.
44. Domoki, F., Bari, F., Nagy, K., Busija, D. W., and Siklos, L. 2004. Diazoxide prevents mitochondrial swelling and Ca2+ accumulation in CA1 pyramidal cells after cerebral ischemia in newborn pigs. Brain Res 1019:97-104.
45. Dunwiddie, T. V., and Lynch, G. 1979. The relationship between extracellular calcium concentrations and the induction of hippocampal long-term potentiation. Brain Res 169:103-110.
46. Gulyaeva, N. V., Kudryashov, I. E., and Kudryashova, I. V. 2003. Caspase activity is essential for long-term potentiation. J Neurosci Res 73:853-864.
47. Kobayashi, K., Ishii, S., Kume, K., Takahashi, T., Shimizu, T., and Manabe, T. 1999. Platelet-activating factor receptor is not required for long-term potentiation in the hippocampal CA1 region. Eur J Neurosci 11:1313-1316.
48. Eliseev, R. A., Vanwinkle, B., Rosier, R. N., and Gunter, T. E. 2004. Diazoxide-mediated preconditioning against apoptosis involves activation of cAMP-response element-binding protein (CREB) and NFkappaB. J Biol Chem 279:46748-46754.
49. Farkas, E., Institoris, A., Domoki, F., Mihaly, A., Luiten, P. G., and Bari, F. 2004. Diazoxide and dimethyl sulphoxide prevent cerebral hypoperfusion-related learning dysfunction and brain damage after carotid artery occlusion. Brain Res 1008:252-260.
50. Chen, H. S., Pellegrini, J. W., Aggarwal, S. K., Lei, S. Z., Warach, S., Jensen, F. E., and Lipton, S. A. 1992. Open-channel block of N-methyl-D-aspartate (NMDA) responses by memantine: therapeutic advantage against NMDA receptor-mediated neurotoxicity. J Neurosci 12:4427-4436.
51. Areosa Sastre, A., McShane, R., and Sherriff, F. 2004. Memantine for dementia. Cochrane Database Syst Rev:CD003154.
52. Chemer, M., Masliah, E., Ellis, R. J., Marcotte, T. D., Moore, D. J., Grant, I., and Heaton, R. K. 2002. Neurocognitive dysfunction predicts postmortem findings of HIV encephalitis. Neurology 59:1563-1567.
53. Perry, S. W., Norman, J.P., Litzburg, A., and Gelbard, H. A. 2004. Antioxidants are required during the early critical period, but not later, for neuronal survival. J Neurosci Res 78:485-492.
54. Liang H W, Xia Q, Bruce I C. Reactive oxygen species mediate the neuroprotection conferred by a mitochondrial ATP-sensitive potassium channel opener during ischemia in the rat hippocampal slice. Brain Res. 2005 May 3; 1042(2):169-75.
55. Eduardo Marban. The surprising role of vascular KATP channels in vasospastic angina. J Clin Invest, July 2002, Volume 110, Number 2, 153-154
56. Otani H, Okada T, Fujiwara H, Uchiyama T, Sumida T, Kido M, Imamura H. Combined pharmacological preconditioning with a G-protein-coupled receptor agonist, a mitochondrial KATP channel opener and a nitric oxide donor mimics ischaemic preconditioning. Clin Exp Pharmacol Physiol. 2003 September; 30(9):684-93.
57. Busija D W, Katakam P, Rajapakse N C, K is B, Grover G, Domoki F, Bari F. Effects of ATP-sensitive potassium channel activators diazoxide and BMS-191095 on membrane potential and reactive oxygen species production in isolated piglet mitochondria. Brain Res Bull. 2005 Jul. 30; 66(2):85-90.
58. K is B, Nagy K, Snipes J A, Rajapakse N C, Horiguchi T, Grover G J, Busija D W. The mitochondrial K(ATP) channel opener BMS-191095 induces neuronal preconditioning. Neuroreport. 2004 Feb. 9; 15(2):345-9.
59. Dunwiddie and Lynch, 1979, The relationship between extracellular calcium concentrations and the induction of hippocampal long-term potentiation. Brain Res 169:103-110.
60. Choi, 1985. Glutamate neurotoxicity in cortical cell culture is calcium dependent. Neurosci Lett.; 58(3):293-7). After entering the dendrites and spines, calcium in the cytosol activates a myriad of enzymes and signaling cascades that are critical for LTP, including calmodulin-dependent kinase II (CaMKII) that strengthens synaptic transmission via phosphorylation and recruitment of AMPA receptors
61. Malenka et al., 1989, An essential role for postsynaptic calmodulin and protein kinase activity in long-term potentiation. Nature. 1989 Aug. 17; 340(6234):554-7.
62. Derkach and Soderling T R. 1999. Ca2+/calmodulin-kinase II enhances channel conductance of alpha-amino-3-hydroxy-5-methyl-4-isoxazolepropionate type glutamate receptors. Proc Natl Acad Sci USA. 96(6):3269-74.
63. Hayashi et al., 2000 Driving AMPA receptors into synapses by LTP and CaMKII: requirement for GluR1 and PDZ domain interaction. Science. Mar. 24; 287(5461):2262-7.
64. Shuttleworth C W, Brennan A M, Connor J A. 2003. NAD(P)H fluorescence imaging of postsynaptic neuronal activation in murine hippocampal slices. J. Neurosci. 23(8):3196-208.
65. Kann O, Schuchmann S, Buchheim K, Heinemann U. 2003 Coupling of neuronal activity and mitochondrial metabolism as revealed by NAD(P)H fluorescence signals in organotypic hippocampal slice cultures of the rat. Neuroscience. 119(1):87-100.
66. Hayakawa Y, Nemoto T, Ino M, Kasai H.2005 Rapid Ca2+-dependent increase in oxygen consumption by mitochondria in single mammalian central neurons. Cell Calcium. 37(4):359-70.
67. White R J, Reynolds I J. 1996. Mitochondrial depolarization in glutamate-stimulated neurons: an early signal specific to excitotoxin exposure. J. Neurosci. 16(18):5688-97.
68. Stout A K, Raphael H M, Kanterewicz B I, Klann E, Reynolds I J. 1998. Glutamate-induced neuron death requires mitochondrial calcium uptake. Nat. Neurosci. 1(5):366-73.
69. Rintoul G L, Filiano A J, Brocard J B, Kress G J, Reynolds I J. 2003 Glutamate decreases mitochondrial size and movement in primary forebrain neurons. J. Neurosci. August 27; 23(21):7881-8.
70. Wu K, Aoki C, Elste A, Rogalski-Wilk A A, Siekevitz P. 1997-1998. The synthesis of ATP by glycolytic enzymes in the postsynaptic density and the effect of endogenously generated nitric oxide. Proc Natl Acad Sci USA. 1997 Nov. 25; 94(24):13273-8. Erratum in: Proc Natl Acad Sci USA 1998 Mar. 3; 95(5):2714.
71. Neuhoff H, Sassoe-Pognetto M, Panzanelli P, Maas C, Witke W, Kneussel M. 2005 The actin-binding protein profilin I is localized at synaptic sites in an activity-regulated manner. Eur J. Neurosci.; 21(1):15-25.
72. Chan S L, Mattson M P. 1999. Caspase and calpain substrates: roles in synaptic plasticity and cell death. J Neurosci Res. 58(1):167-90.
73. Gulyaeva N V, Kudryashov I E, Kudryashova I V. 2003 Caspase activity is essential for long-term potentiation. J Neurosci Res. September 15; 73(6):853-64.
74. Perry, S. W., Litzburg, A., Bellizzi, M. J., and H. A. Gelbard. 2005 HIV-1 transactivation of transcription protein induces mitochondrial hyperpolarization and synaptic stress leading to apoptosis. J. Immunol. 174(7):4333-4344.
75. Mattson M P, Duan W. 1999 “Apoptotic” biochemical cascades in synaptic compartments: roles in adaptive plasticity and neurodegenerative disorders. J Neurosci Res. 58(1):152-66.
76. Chan S L, Mattson M P. 1999 Caspase and calpain substrates: roles in synaptic plasticity and cell death. J Neurosci Res. 58(1):167-90].
77. Wang P, Barks J D, Silverstein F S. 1999. Tat, a human immunodeficiency virus-1-derived protein, augments excitotoxic hippocampal injury in neonatal rats. Neuroscience. 88(2):585-97.
78. Dabrowski, M, Larsen, T, Ashcroft, F M, Bondo Hansen, J, and P. Wahl. 2003. Potent and selective activation of the pancreatic beta-cell type K(ATP) channel by two novel diazoxide analogues. Diabetologia. (10):1375-82.
79. Dou et al., 2003. Neuroprotective activities of sodium valproate in a murine model of HIV-1 encephalitis. J. Neurosci. 23:9162-9170.
80. Nelson et al., 2005. Coregistration of quantitative proton magnetic resonance spectroscopic imaging with neuropathological and neurophysiological analyses defines the extent of neuronal impairments in murine HIV-1 encephalitis. J. Neurosci. Res., 80(4):562-75.
81. Dou et al., 2005. Neuroprotective mechanisms of lithium in murine human immunodeficiency virus-1 encephalitis. J. Neurosci. 25(37):8375-85.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A method of preventing HIV-1 associated dendritic pathology in a brain cell, comprising contacting the cell with a therapeutically effective dose of a mitochondrial ATP-sensitive potassium channel agonist.

2. The method of claim 1, wherein the mitochondrial ATP-sensitive potassium channel agonist is a compound having the structure of Formula II

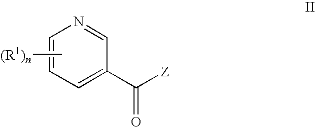

3. The method of claim 2, wherein the mitochondrial ATP-sensitive potassium channel agonist is nicorandil.

4. A method of preventing HIV-1 associated dendritic pathology in a brain cell, comprising contacting the cell with a therapeutically effective dose of an inhibitor of succinate dehydrogenase.

5. A method of preventing HIV-1 associated dendritic pathology in a brain cell, comprising contacting the cell with a therapeutically effective dose of a stimulator of production of reactive oxygen species.

6. A model for the study of HIV-1 associated dendritic pathology, comprising
a) contacting a hippocampal slice with platelet-activating factor; and
b) stimulating the hippocampal slice of step a) with high frequency stimulation.

7. A method of screening for inhibitors of HIV-1 associated dendritic pathology in a brain cell, comprising:
a) contacting a hippocampal slice with the putative inhibitor compound;
b) contacting the hippocampal slice of step a) with platelet-activating factor;
c) stimulating the hippocampal slice of step b) with high frequency stimulation; and
d) detecting a reduction in HIV-1 associated dendritic pathology in a cell in the hippocampal slice contacted with the putatitve inhibitor, a reduction in dendritic pathology, compared to a hippocampal slice not receiving the putative inhibitor, indicating that the compound is an inhibitor of dendritic pathology.
